(19) United States
(12) Patent Application Publication
(10) Pub. No.: US 2024/0309007 A1
(43) Pub. Date: Sep. 19, 2024

(54) CRYSTALLINE FORMS OF 3-{4-[(2R)-2-AMINOPROPOXY]PHENYL}-N-[(1R)- 1-(3-FLUOROPHENYL) ETHYL]IMIDAZO[1,2-B]PYRIDAZIN-6-AMINE AND SALTS THEREOF

(71) Applicant: ANHEART THERAPEUTICS (HANGZHOU) CO., LTD., Hangzhou, Zhejiang (CN)

(72) Inventors: Fenger Zhou, Shanghai (CN); Doug Dagang Chen, Shanghai (CN); Ping Li, Tianjin (CN); Jingwen Lu, Tianjiin (CN); Jun Xue, Tianjin (CN)

(21) Appl. No.: 18/575,414

(22) PCT Filed: Jul. 1, 2021
(86) PCT No.: PCT/CN2021/104090

§ 371 Date: Dec. 29, 2023

(51) Int. Cl. C07D 487/04; A61K 31/5025

(52) U.S. Cl. CPC C07D 487/04; A61K 31/5025

(57) ABSTRACT

This disclosure relates to crystalline forms of 3-{4-[(2R)-2-aminopropoxy]phenyl}-N-[(1R)-1-(3-fluorophenyl) ethyl]imidazo[1,2-b]pyridazin-6-amine and its salts, as well as methods of preparing and using such crystalline forms.

### TECHNICAL FIELD

This disclosure relates to crystalline forms of 3-{4-[(2R)-2-aminopropoxy]phenyl}-N-[(1R)-1-(3-fluorophenyl)ethyl]imidazo[1,2-b]pyridazin-6-amine and its salts, as well as methods of preparing and using such crystalline forms.

### BACKGROUND

3-{4-[(2R)-2-Aminopropoxy]phenyl}-N-[(1R)-1-(3-fluorophenyl)ethyl]-imidazo[1,2-b]pyridazin-6-amine (Compound 1) is a known ROS1 receptor tyrosine kinase inhibitor and neurotrophic tyrosine receptor kinase (NTRK) inhibitor, and has the following chemical structure:

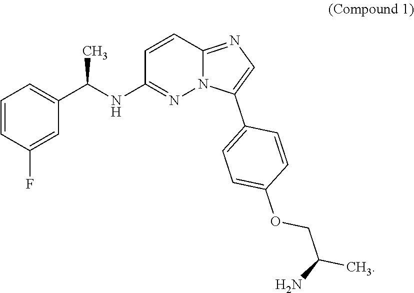 [SMILES: C[C@@H](N)COc1ccc(-c2cnc3ccc(N[C@H](C)c4cccc(F)c4)nn23)cc1]

The ROS1 gene encodes a receptor tyrosine kinase which was discovered as a human ortholog of the cancer gene product v-ros of avian sarcoma virus UR2 (University of Rochester tumor virus 2). The ROS1 fusion gene resulting from the chromosomal rearrangement containing the ROS1 gene and the subsequent fusion of the ROS1 gene to another gene was discovered in a glioblastoma cell line U118MG. In the U118MG cells, a gene encoding a Golgi protein FIG (fused in glioblastoma) is fused with the ROS1 gene to form a gene encoding FIG-ROS1 fusion protein. The fusion between FIG and ROS1 causes structural change that constitutively activates ROS1 kinase enzyme activity, and the FIG-ROS1 fusion protein has cell transformation activity and tumorigenic activity mediated by the activation of the ROS1 signaling pathway involving STAT3, ERK, and SHP2.

The chromosomal translocation of the ROS1 gene has also been identified in a non-small cell lung cancer cell line HCC78 and clinical specimens of lung cancers. The fusion gene of the SLC34A2 gene and the ROS1 gene has been reported in the HCC78 cells, while the presence of the transmembrane protein-encoding CD74-ROS1 fusion gene of the CD74 gene and the ROS1 gene has been reported in non-small cell lung cancer patient specimens. The fusion gene of the FIG gene and the ROS1 gene has been found in 2 out of 23 patient specimens of bile duct cancer.

The large-scale screening of patient specimens using FISH (fluorescent in situ hybridization) has identified fusion genes of the ROS1 gene with SDC, CD74, EZR, SLC34A2, LRIG3, or TPM3. Any of the ROS1 fusion genes SDC-ROS1, CD74-ROS1, EZR-ROS1, SLC34A2-ROS1, LRIG3-ROS1, and TPM3-ROS1 have been detected in 13 out of 1476 non-small cell lung cancer patient specimens.

Likewise, the large-scale screening of non-small cell lung cancer patient specimens using FISH has found the ROS1 fusion gene in 18 out of 1073 cases. In addition, analysis using patient specimens has showed that the ROS1 gene is highly expressed in brain tumor.

ROS1 has been shown to be activated in cancer expressing the ROS1 fusion gene (e.g., non-small cell lung cancer, bile duct cancer, or brain tumor). Thus, a drug that inhibits ROS1 kinase activity can block the downstream of the ROS1 pathway, i.e., STAT3, ERK, SHP2, which contribute the tumor growth and tumor cell survival. Therefore, ROS1 kinase inhibitor is expected to be useful as a therapeutic drug for cancer. Compounds such as crizotinib, TAE684, pyrazole derivatives, and aminopyrazine derivatives have been reported to have an inhibitory effect on ROS1 kinase enzyme activity.

Neurotrophic tyrosine receptor kinase, also called tropomyosin-related kinase (Trk), is a high-affinity receptor that is activated by a soluble growth factor called neurotrophin (NT). The NTRK receptor family has three members: NTRK1 (also called TrkA), NTRK2 (also called TrkB), and NTRK3 (also called TrkC).

NT includes a plurality of proteins as follows: a nerve growth factor (NGF) which activates NTRK1, a brain-derived neurotrophic factor (BDNF) and NT-4/5 which activate NTRK2, and NT3 which activates NTRK3. Each NTRK receptor contains an extracellular domain (ligand-binding site), a transmembrane domain, and an intracellular domain (containing a kinase domain). Upon binding to a ligand, each kinase catalyzes autophosphorylation and then activates the downstream signal transduction pathway.

NTRK is widely expressed in nerve tissues during their development period and plays an important role for the maintenance and survival of these cells. The previous study shows that NTRK plays an important role in both the development and function of the nervous system.

A large number of references state that NTRK signal transduction is associated 10 with cancer. For example, NTRK exists at a low expression level in regions other than the nervous system in adult humans, whereas the expression of NTRK is increased at the late stage of prostate cancer. In normal prostate tissues and androgen-dependent prostate tumor at the early state, NTRK1 is expressed only at a low level or an undetectable level, but neither NTRK2 nor NTRK3 is expressed. In androgen-independent prostate cancer at the late stage, however, all isoforms of the NTRK receptors and their ligands are overexpressed. The evidence shows that these late-stage prostate cancer cells depend on NTRK for their tumor survival. Thus, NTRK inhibitors may induce apoptosis for androgen-independent prostate cancer. In addition, recent references also show that the overexpression, activation, amplification, fusion gene formation, or mutation of NTRK is related to neuroblastoma, secretory breast cancer, colorectal cancer, ovary cancer, head and neck cancer, pancreatic cancer, and melanoma.

Selective NTRK tyrosine kinase inhibitors have been reported, including CEP-751, CEP-701, indolocarbazole compounds, oxindole compounds, pyrazolyl condensed-ring compounds, isothiazole compounds, and other various compounds.

### SUMMARY

This disclosure is based on the unexpected discover that certain crystalline forms of Compound 1 or its salts possesses superior physical properties (e.g., physical and chemical stability).

In one aspect, this disclosure features a crystalline form A of 3-{4-[(2R)-2-aminopropoxy]phenyl}-N-[(1R)-1-(3-fluorophenyl)ethyl]imidazo[1,2-b]pyridazin-6-amine (Compound 1) adipate, in which the crystalline form A has a tetragonal crystal system, the space group is P4₁2₁2, and the unit cell parameters are a=b=9.63 (1) Å, c=61.14 (2) Å, α=β=γ=90°, and V=5666 (5) Å³.

In another aspect, this disclosure features a method of preparing the crystalline form A of Compound 1 adipate. The method includes mixing an amorphous form of Compound 1 adipate with a solvent; and adding an anti-solvent into the mixture to obtain the crystalline form A of Compound 1 adipate.

In another aspect, this disclosure features a crystalline form B of 3-{4-[(2R)-2-aminopropoxy]phenyl}-N-[(1R)-1-(3-fluorophenyl)ethyl]imidazo[1,2-b]pyridazin-6-amine (Compound 1) adipate, in which the crystalline form B exhibits an X-ray powder diffraction (XRPD) pattern that comprises at least one diffraction peak having a diffraction angle 20 selected from the group consisting of 5.2±0.2°, 7.2±0.2°, and 20.9±0.2° obtained by using CuKα radiation.

In another aspect, this disclosure features a method of preparing the crystalline form B of Compound 1 adipate. The method includes dissolving an amorphous form of Compound 1 adipate in a solvent comprising dichloromethane and methanol to form a solution; and evaporating the solvent to obtain the crystalline form B of Compound 1 adipate.

In another aspect, this disclosure features a crystalline form C of 3-{4-[(2R)-2-aminopropoxy]phenyl}-N-[(1R)-1-(3-fluorophenyl)ethyl]imidazo[1,2-b]pyridazin-6-amine (Compound 1) adipate, in which the crystalline form C exhibits an X-ray powder diffraction (XRPD) pattern that comprises at least one diffraction peak having a diffraction angle 2θ selected from the group consisting of 5.7±0.2°, 21.0±0.2°, and 23.2±0.2° obtained by using CuKα radiation.

In another aspect, this disclosure features a method of preparing the crystalline form C of Compound 1 adipate. The method includes dissolving an amorphous form of Compound 1 adipate in ethanol to form a solution; adding acetone into the solution; and removing ethanol and acetone by evaporation to obtain the crystalline form C of Compound 1 adipate.

In another aspect, this disclosure features a crystalline form D of 3-{4-[(2R)-2-aminopropoxy]phenyl}-N-[(1R)-1-(3-fluorophenyl)ethyl]imidazo[1,2-b]pyridazin-6-amine (Compound 1) adipate, in which the crystalline form D exhibits an X-ray powder diffraction (XRPD) pattern that comprises at least one diffraction peak having a diffraction angle 2θ selected from the group consisting of 4.9±0.2°, 19.4±0.2°, and 21.6±0.2° obtained by using CuKα radiation.

In another aspect, this disclosure features a method of preparing the crystalline form D of Compound 1 adipate. The method includes dissolving an amorphous form of Compound 1 adipate in dimethylacetamide to forming a solution; adding acetone into the solution; and removing dimethylacetamide and acetone by evaporation to obtain the crystalline form D of Compound 1 adipate.

In another aspect, this disclosure features a crystalline form A of 3-{4-[(2R)-2-aminopropoxy]phenyl}-N-[(1R)-1-(3-fluorophenyl)ethyl]imidazo[1,2-b]pyridazin-6-amine (Compound 1) free base, in which the crystalline form A exhibits an X-ray powder diffraction (XRPD) pattern that comprises at least one diffraction peak having a diffraction angle 2θ selected from the group consisting of 8.5±0.2°, 12.7±0.2°, and 19.1±0.2° obtained by using CuKα radiation.

In another aspect, this disclosure features a method of preparing the crystalline form A of Compound 1 free base. The method includes mixing a base with a solution containing Compound 1 hydrochloride in water and an alcohol to obtain the crystalline form A of Compound 1 free base.

In another aspect, this disclosure features a crystalline form B of 3-{4-[(2R)-2-aminopropoxy]phenyl}-N-[(1R)-1-(3-fluorophenyl)ethyl]imidazo[1,2-b]pyridazin-6-amine (Compound 1) free base, in which the crystalline form B exhibits an X-ray powder diffraction (XRPD) pattern that comprises at least one diffraction peak having a diffraction angle 2θ selected from the group consisting of 6.1±0.2°, 9.4±0.2°, and 21.3±0.2° obtained by using CuKα radiation.

In another aspect, this disclosure features a method of preparing the crystalline form B of Compound 1 free base. The method includes dispersing a crystalline form A of Compound 1 free base in dichloromethane to forming a dispersion; and stirring the dispersion at a temperature from about 45° C. to about 55° C. (e.g., about 50° C.°) to obtain the crystalline form B of Compound 1 free base.

In another aspect, this disclosure features a crystalline form C of 3-{4-[(2R)-2-aminopropoxy]phenyl}-N-[(1R)-1-(3-fluorophenyl)ethyl]imidazo[1,2-b]pyridazin-6-amine (Compound 1) free base, in which the crystalline form C exhibits an X-ray powder diffraction (XRPD) pattern that comprises at least one diffraction peak having a diffraction angle 2θ selected from the group consisting of 18.6±0.2°, 20.2±0.2°, and 21.1±0.2° obtained by using CuKα radiation.

In another aspect, this disclosure features a method of preparing the crystalline form C of Compound 1 free base. The method includes dissolving a crystalline form A of Compound 1 free base in dichloromethane to forming a solution; and removing dichloromethane by evaporation to obtain the crystalline form C of Compound 1 free base.

In another aspect, this disclosure features a crystalline form D of 3-{4-[(2R)-2-aminopropoxy]phenyl}-N-[(1R)-1-(3-fluorophenyl)ethyl]imidazo[1,2-b]pyridazin-6-amine (Compound I) free base, in which the crystalline form D exhibits an X-ray powder diffraction (XRPD) pattern that comprises at least one diffraction peak having a diffraction angle 2θ selected from the group consisting of 8.6±0.2°, 18.4±0.2°, and 20.9±0.2° obtained by using CuKα radiation.

In another aspect, this disclosure features a method of preparing the crystalline form D of Compound 1 free base. The method includes dissolving a crystalline form A of Compound 1 free base in methanol to forming a solution; adding methyl tert-butyl ether to the solution; and removing methanol and methyl tert-butyl ether by evaporation to obtain the crystalline form D of Compound 1 free base.

In another aspect, this disclosure features a pharmaceutical composition that includes at least one crystalline form described herein; and a pharmaceutically acceptable carrier.

In still another aspect, this disclosure features a method of treating cancer. The method includes administering to a subject in need thereof a therapeutically effective amount of the pharmaceutical composition described herein.

Embodiments described herein can have one or more of the following advantages.

In some embodiments, certain crystalline forms described herein have relatively low hygroscopicity. For example, the weight gains of the crystalline forms A and D of Compound 1 adipate at 80% RH are 0.235% and 0.34%, respectively. As another example, the weight gains of the crystalline forms A, B, and C of Compound 1 free base at 80% RH are 0.1%, 0.005%, and 0.107%, respectively. Hygroscopicity affects the stability of drug substances, flowability and uniformity during the formulation process, thus affecting the quality of drug products. Hygroscopicity also affects the preparation, storage and post-treatment of drugs. Crystalline forms with low hygroscopicity are not demanding on storage conditions, which reduces the cost of storage and quality control.

In some embodiments, certain crystalline forms described herein have superior 15 physical stability. For example, the crystalline form A of Compound 1 adipate remains unchanged in the crystalline for at least two weeks when stored in air under the conditions of 40° C./75% RH. Superior physical stability is of great importance to the drug development. There are numerous processes (including storage, transportation and formulation processes) during the manufacturing of a drug product. These processes are often under stress conditions, which can be caused by the collision of drug substance in storage and transportation, the wet granulation process in drug production, the seasonal and regional climate differences, and weather factors. High temperature and high humidity are the most common stress condition. Crystalline form transformation during these processes can lead to changes in the absorption of the drug, or cause toxicity and side effects. The crystalline form A of Compound 1 adipate has superior physical stability, which ensures consistent and controllable quality of the drug substance and drug product, minimizes toxicity caused by crystal transformation and ensures the therapeutic effect of the drug.

In some embodiments, certain crystalline forms described herein have superior chemical stability. For example, when stored in air under the conditions of 40° C./75% RH for two weeks, the purity of the crystalline form A of Compound 1 adipate is essentially unchanged. Chemical purity is of great significance for ensuring drug efficacy and safety, and for preventing the occurrence of adverse effects. If the drug contains impurities higher than limit, its physicochemical properties and drug appearance may change, and the stability may be affected. The increase in impurities also leads to lowered active ingredient content, reduced drug activity, and/or increased toxicity and side effects of the drug products. The crystalline form A of Compound 1 adipate has little change in purity after storage and are non-degradable, which effectively minimize the potential risk of reduction in drug purity, reduction in drug efficacy, and increased toxicity.

In some embodiments, certain crystalline forms described herein (e.g., the crystalline forms A-C of Compound 1 adipate and the crystalline forms A-C of Compound 1 free base) have almost no residue organic solvent. In general, if the residual organic solvent in a compound exceeds the relevant standards, the compound may not be used as a drug substance as many organic solvents are harmful to human and environment. Therefore, to ensure drug safety and product quality, it is necessary to minimize the residual organic solvent of a drug substance.

Other features, objects, and advantages will be apparent from the description and the claims.

### DESCRIPTION OF DRAWINGS

FIG. 1 shows an X-ray powder diffraction (XRPD) pattern of crystalline form A of Compound 1 adipate.

FIG. 2 shows an TGA curve of crystalline form A of Compound 1 adipate.

FIG. 3 shows an DSC curve of crystalline form A of Compound 1 adipate.

FIG. 4 shows an DVS curve of crystalline form A of Compound 1 adipate.

FIG. 5 shows an XRPD pattern of crystalline form B of Compound 1 adipate.

FIG. 6 shows an TGA curve of crystalline form B of Compound 1 adipate.

FIG. 7 shows an DSC curve of crystalline form B of Compound 1 adipate.

FIG. 8 shows an DVS curve of crystalline form B of Compound 1 adipate.

FIG. 9 shows an XRPD pattern of crystalline form C of Compound 1 adipate.

FIG. 10 shows an TGA curve of crystalline form C of Compound 1 adipate.

FIG. 11 shows an DSC curve of crystalline form C of Compound 1 adipate.

FIG. 12 shows an DVS curve of crystalline form C of Compound 1 adipate.

FIG. 13 shows an XRPD pattern of crystalline form D of Compound 1 adipate.

FIG. 14 shows an TGA curve of crystalline form D of Compound 1 adipate.

FIG. 15 shows an DSC curve of crystalline form D of Compound 1 adipate.

FIG. 16 shows an DVS curve of crystalline form D of Compound 1 adipate.

FIG. 17 shows an XRPD pattern of crystalline form A of Compound 1 free base.

FIG. 18 shows an TGA curve of crystalline form A of Compound 1 free base.

FIG. 19 shows an DSC curve of crystalline form A of Compound 1 free base.

FIG. 20 shows an DVS curve of crystalline form A of Compound 1 free base.

FIG. 21 shows an XRPD pattern of crystalline form B of Compound 1 free base.

FIG. 22 shows an TGA curve of crystalline form B of Compound 1 free base.

FIG. 23 shows an DSC curve of crystalline form B of Compound 1 free base.

FIG. 24 shows an DVS curve of crystalline form B of Compound 1 free base.

FIG. 25 shows an XRPD pattern of crystalline form C of Compound 1 free base.

FIG. 26 shows an TGA curve of crystalline form C of Compound 1 free base.

FIG. 27 shows an DSC curve of crystalline form C of Compound 1 free base.

FIG. 28 shows an DVS curve of crystalline form C of Compound 1 free base.

FIG. 29 shows an XRPD pattern of crystalline form D of Compound 1 free base.

FIG. 30 shows an TGA curve of crystalline form D of Compound 1 free base.

FIG. 31 shows an DSC curve of crystalline form D of Compound 1 free base.

FIG. 32 shows an DVS curve of crystalline form D of Compound 1 free base.

Like reference symbols in the various drawings indicate like elements.

### DETAILED DESCRIPTION

This disclosure generally relates to crystalline forms of 3-{4-[(2R)-2-aminopropoxy]phenyl}-N-[(1R)-1-(3-fluorophenyl)ethyl]imidazo[1,2-b]pyridazin-6-amine (Compound 1) and its salts, as well as methods of preparing and using such crystalline forms.

### Crystalline Forms and Methods of Preparation

### Crystalline Form A of Compound 1 Adipate

In some embodiments, this disclosure features a crystalline form A of Compound 1 adipate. In general, the crystalline form A of Compound 1 adipate can be characterized by related crystal system and related unit cell parameters. In some embodiments, the crystalline form A has a tetragonal crystal system, the space group is P4₁2₁2, and the unit cell parameters are a=b=9.63 (1) Å, c=61.14 (2) Å, α=β=γ=90°, and V=5666 (5) Å³. It is believed that the crystalline form A of Compound 1 adipate is an anhydrate.

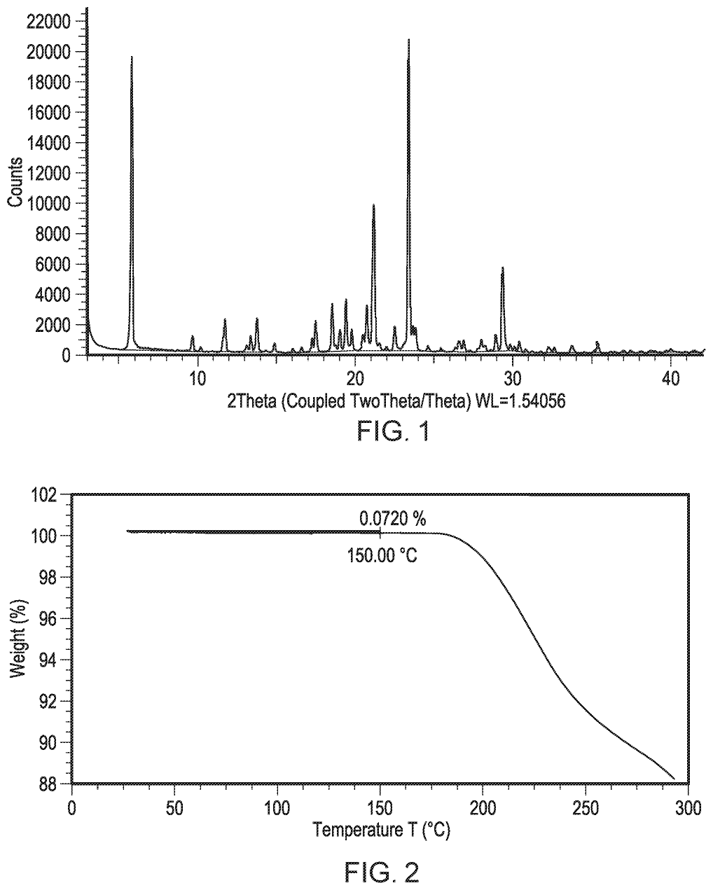

In some embodiments, the crystalline form A of Compound 1 adipate exhibits an XRPD pattern that includes at least one (e.g., two or three) diffraction peak having a diffraction angle 2θ selected from the group consisting of 5.8±0.2°, 21.1±0.2°, and 23.3±0.2° obtained by using CuKα radiation. In some embodiments, the XRPD pattern of the crystalline form A of Compound 1 adipate further includes at least one (e.g., two or three) diffraction peak having a diffraction angle 2θ selected from the group consisting of 18.5±0.2°, 19.4±0.2°, and 29.2±0.2° obtained by using CuKα radiation. In some embodiments, the XRPD pattern of the crystalline form A of Compound 1 adipate further includes at least one (e.g., two or three) diffraction peak having a diffraction angle 2θ selected from the group consisting of 11.7±0.2°, 13.7±0.2°, and 20.7±0.2° obtained by using CuKα radiation. In some embodiments, the XRPD pattern of the crystalline form A of Compound 1 adipate includes at least one (e.g., 2, 3, 4, 5, 6, 7, 9, 10, 11, 12, 13, 14, 15, or all) of the diffraction peaks in Table 3 below. In some embodiments, the XRPD pattern of the crystalline form A of Compound 1 adipate is substantially as depicted in FIG. 1.

In some embodiments, the crystalline form A of Compound 1 adipate can have a relatively high solubility in an aqueous buffer solution with a pH of 1.0 or a simulated gastric fluid (SGF), which suggests that it can be readily dissolved in the gastric fluid in stomach. For example, the crystalline form A of Compound 1 adipate can have a solubility of from at least about 10 mg/mL (e.g., at least about 12 mg/mL, at least about 14 mg/mL, or at least about 15 mg/mL) to at most about 25 mg/mL (e.g., at most about 24 mg/mL, at most about 22 mg/mL, or at most about 20 mg/mL) in an aqueous buffer solution with a pH of 1.0 or an SGF.

In general, the crystalline form A of Compound 1 adipate has superior physical and/or chemical stability (e.g., at an elevated temperature such as 40° C., in an environment with a relative high humidity such as at least 60% RH, and/or under exposure to light). For example, the crystalline form A of Compound 1 adipate can be stable at room temperature in a sealed container for at least 36 months.

In general, the crystalline form A of Compound 1 adipate has superior solubility. For example, the crystalline form A of Compound 1 adipate can have a solubility of at least about 15 mg/mL (e.g., at least about 17 mg/mL) in a simulated gastric fluid or in a buffer solution with a pH of 1. In general, the crystalline form A of Compound 1 adipate has very low or nearly no hygroscopicity.

In some embodiments, the crystalline form A of Compound 1 adipate can be prepared by a method that includes the following steps: (1) mixing an amorphous form of Compound 1 adipate with a solvent; and (2) adding an anti-solvent into the mixture to obtain the crystalline form A of Compound 1 adipate. In general, upon addition of the anti-solvent into the mixture, a white crystal appears in from about 5 minutes to about 12 hours. In some embodiments, when no crystal appears upon addition of the anti-solvent into the mixture, the solvent and anti-solvent can be removed by evaporation (e.g., by blow drying) to obtain the crystalline form A of Compound 1 adipate.

In some embodiments, the solvent suitable for preparing the crystalline form A of Compound 1 adipate can include an alcohol (e.g., ethanol or isopropanol), a sulfoxide (e.g., dimethylsulfoxide (DMSO)), or an amide (e.g., dimethylformamide (DMF) or dimethylacetamide (DMAc)).

In some embodiments, the solvent suitable for preparing the crystalline form A of Compound 1 adipate can include a hydrocarbon (e.g., heptane or toluene), an ether (e.g., tetrahydrofuran (THF) or methyl tert-butyl ether), a nitrile (e.g., acetonitrile), a ketone (e.g., acetone), an ester (e.g., ethyl acetate), or water.

### Crystalline Form B of Compound 1 Adipate

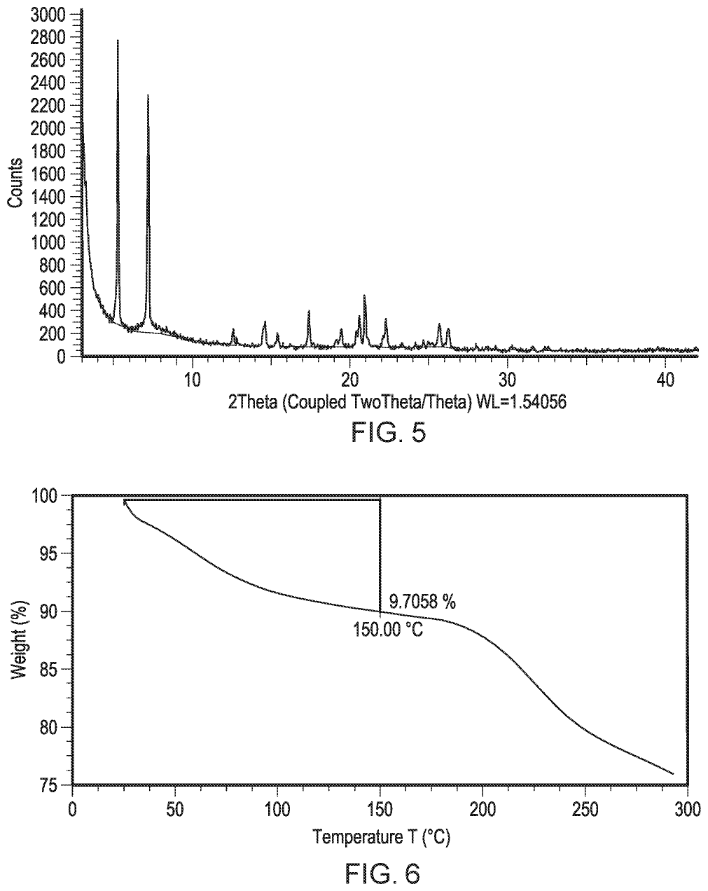

In some embodiments, this disclosure features a crystalline form B of Compound 1 adipate. In some embodiments, the crystalline form B of Compound 1 adipate exhibits an XRPD pattern that includes at least one (e.g., two or three) diffraction peak having a diffraction angle 2θ selected from the group consisting of 5.2±0.2°, 7.2±0.2°, and 20.9±0.2° obtained by using CuKα radiation. In some embodiments, the XRPD pattern of the crystalline form B of Compound 1 adipate further includes at least one (e.g., two or three) diffraction peak having a diffraction angle 2θ selected from the group consisting of 17.3±0.2°, 20.5±0.2°, and 22.2±0.2° obtained by using CuKα radiation. In some embodiments, the XRPD pattern of the crystalline form B of Compound 1 adipate further includes at least one (e.g., two or three) diffraction peak having a diffraction angle 2θ selected from the group consisting of 14.5±0.2°, 25.7±0.2°, and 26.2±0.2° obtained by using CuKα radiation. In some embodiments, the XRPD pattern of the crystalline form B of Compound 1 adipate includes at least one (e.g., 2, 3, 4, 5, 6, 7, 9, 10, or all) of the diffraction peaks in Table 6 below. In some embodiments, the XRPD pattern of the crystalline form B of Compound 1 adipate is substantially as depicted in FIG. 5. It is believed that the crystalline form B of Compound 1 adipate is a hydrate and includes about 3.6 molar water per molar Compound 1 adipate.

In some embodiments, the crystalline form B of Compound 1 adipate can be prepared by a method that includes the following steps: (1) dissolving an amorphous form of Compound 1 adipate in a solvent containing dichloromethane and methanol to form a solution; and (2) evaporating the solvent to obtain the crystalline form B of Compound 1 adipate. In some embodiments, the volume ratio of the dichloromethane and methanol in the solvent used in step (1) can range from about 2:1 to about 1:2 (e.g., about 1:1). In some embodiments, the evaporation in step (2) can be performed by exposing the solution to air under room temperature (e.g., without heating or using rotary evaporation).

### Crystalline Form C of Compound 1 Adipate

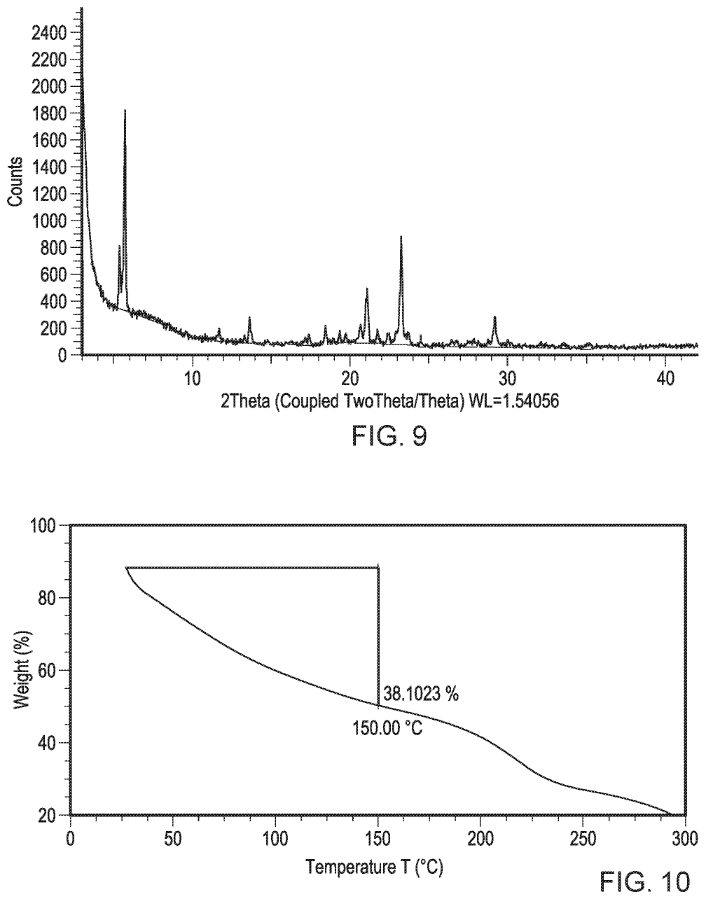

In some embodiments, this disclosure features a crystalline form C of Compound 1 adipate. In some embodiments, the crystalline form C of Compound 1 adipate exhibits an XRPD pattern that includes at least one (e.g., two or three) diffraction peak having a diffraction angle 2θ selected from the group consisting of 5.7±0.2°, 21.0±0.2°, and 23.2±0.2° obtained by using CuKα radiation. In some embodiments, the XRPD pattern of the crystalline form C of Compound 1 adipate further includes at least one (e.g., two or three) diffraction peak having a diffraction angle 2θ selected from the group consisting of 5.4±0.2°, 13.6±0.2°, and 29.2±0.2° obtained by using CuKα radiation. In some embodiments, the XRPD pattern of the crystalline form C of Compound 1 adipate further includes at least one (e.g., two or three) diffraction peak having a diffraction angle 2θ selected from the group consisting of 18.4±0.2°, 20.6±0.2°, and 21.7±0.2° obtained by using CuKα radiation. In some embodiments, the XRPD pattern of the crystalline form C of Compound 1 adipate includes at least one (e.g., 2, 3, 4, 5, 6, 7, 9, 10, or all) of the diffraction peaks in Table 7 below. In some embodiments, the XRPD pattern of the crystalline form C of Compound 1 adipate is substantially as depicted in FIG. 9. It is believed that the crystalline form C of Compound 1 adipate is a hydrate and includes about 13 molar water per molar Compound 1 adipate. In general, the crystalline form C of Compound 1 adipate has low hygroscopicity.

In some embodiments, the crystalline form C of Compound 1 adipate can be prepared by a method that includes the following steps: (1) dissolving an amorphous form of Compound 1 adipate in ethanol to form a solution; (2) adding acetone into the solution; and (3) removing ethanol and acetone by evaporation to obtain the crystalline form C of Compound 1 adipate. In some embodiments, the above method can further include stirring the solution after step (2) for an extended period of time, such as at least one hour (e.g., at least 5 hours, at least 10 hours, at least 24 hours). In some embodiments, the evaporation in step (3) can be performed by blow drying the solution using nitrogen under room temperature (e.g., without heating or using rotary evaporation).

### Crystalline Form D of Compound 1 Adipate

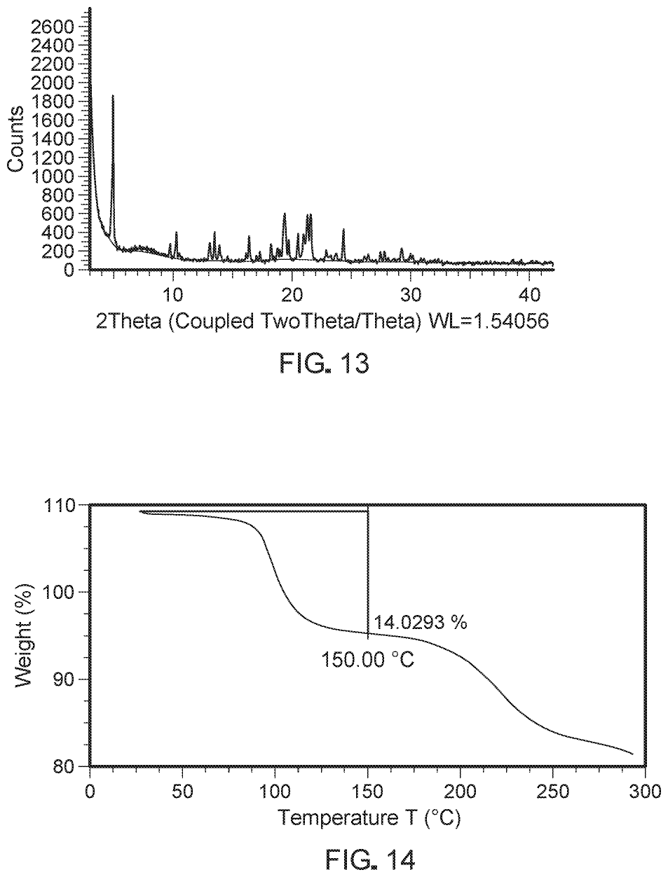

In some embodiments, this disclosure features a crystalline form D of Compound 1 adipate. In some embodiments, the crystalline form D of Compound 1 adipate exhibits an XRPD pattern that includes at least one (e.g., two or three) diffraction peak having a diffraction angle 2θ selected from the group consisting of 4.9±0.2°, 19.4±0.2°, and 21.6±0.2° obtained by using CuKα radiation. In some embodiments, the XRPD pattern of the crystalline form D of Compound 1 adipate further includes at least one (e.g., two or three) diffraction peak having a diffraction angle 2θ selected from the group consisting of 13.5±0.2°, 21.3±0.2°, and 24.3±0.2° obtained by using CuKα radiation. In some embodiments, the XRPD pattern of the crystalline form D of Compound 1 adipate further includes at least one (e.g., two or three) diffraction peak having a diffraction angle 2θ selected from the group consisting of 10.3±0.2°, 16.4±0.2°, and 20.5±0.2° obtained by using CuKα radiation. In some embodiments, the XRPD pattern of the crystalline form D of Compound 1 adipate includes at least one (e.g., 2, 3, 4, 5, 6, 7, 9, 10, 11, 12, 13, 14, or all) of the diffraction peaks in Table 8 below. In some embodiments, the XRPD pattern of the crystalline form D of Compound 1 adipate is substantially as depicted in FIG. 13. It is believed that the crystalline form D of Compound 1 adipate is a DMAc solvate and includes about 1.3 molar DMAc per molar Compound 1 adipate. In general, the crystalline form D of Compound 1 adipate has low hygroscopicity.

In some embodiments, the crystalline form D of Compound 1 adipate can be prepared by a method that includes the following steps: (1) dissolving an amorphous form of Compound 1 adipate in dimethylacetamide to form a solution; (2) adding acetone into the solution; and (3) removing dimethylacetamide and acetone by evaporation to obtain the crystalline form D of Compound 1 adipate. In some embodiments, the above method can further include stirring the solution after step (2) for an extended period of time, such as at least one hour (e.g., at least 5 hours, at least 10 hours, at least 24 hours). In some embodiments, the evaporation in step (3) can be performed by blow drying the solution using nitrogen under room temperature (e.g., without heating or using rotary evaporation).

### Crystalline Form A of Compound 1 Free Base

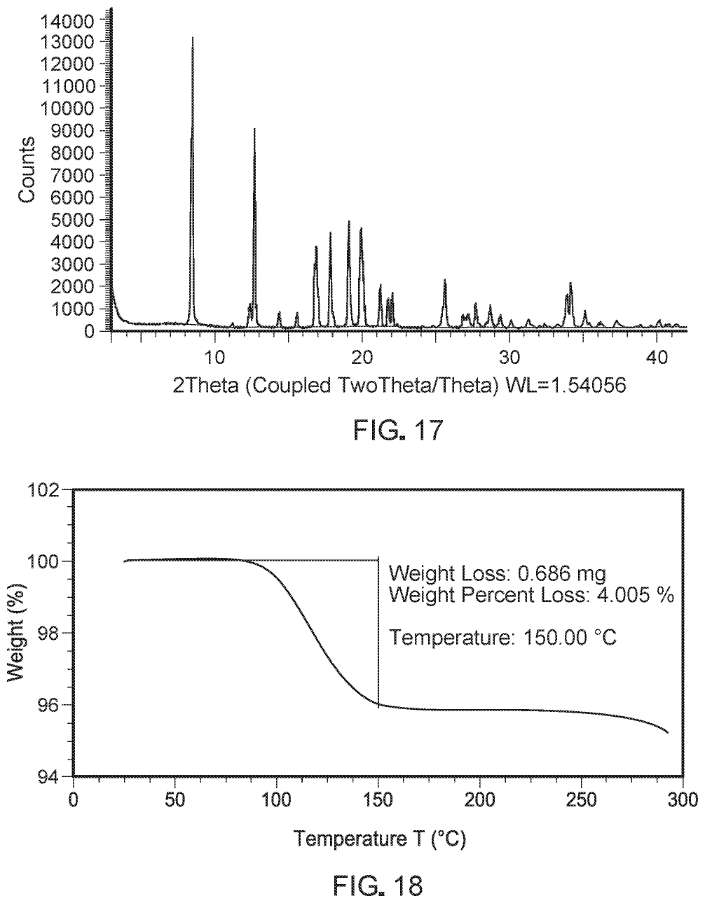

In some embodiments, this disclosure features a crystalline form A of Compound 1 free base. In some embodiments, the crystalline form A of Compound 1 free base exhibits an XRPD pattern that includes at least one (e.g., two or three) diffraction peak having a diffraction angle 2θ selected from the group consisting of 8.5±0.2°, 12.7±0.2°, and 19.1±0.2° obtained by using CuKα radiation. In some embodiments, the XRPD pattern of the crystalline form A of Compound 1 free base further includes at least one (e.g., two or three) diffraction peak having a diffraction angle 2θ selected from the group consisting of 16.9±0.2°, 17.9±0.2°, and 20.0±0.2° obtained by using CuKα radiation. In some embodiments, the XRPD pattern of the crystalline form A of Compound 1 free base further includes at least one (e.g., two or three) diffraction peak having a diffraction angle 2θ selected from the group consisting of 21.3±0.2°, 25.6±0.2°, and 34.1±0.2° obtained by using CuKα radiation. In some embodiments, the XRPD pattern of the crystalline form A of Compound 1 free base includes at least one (e.g., 2, 3, 4, 5, 6, 7, 9, 10, 11, 12, 13, 14, 15, or all) of the diffraction peaks in Table 10 below. In some embodiments, the XRPD pattern of the crystalline form A of Compound 1 free base is substantially as depicted in FIG. 17. It is believed that the crystalline form A of Compound 1 free base is a hydrate and includes about 1 molar water per molar Compound 1 free base.

In general, the crystalline form A of Compound 1 free base has superior physical and/or chemical stability. For example, the crystalline form A of Compound 1 free base can be stable at room temperature in a sealed container for at least 36 months. In addition, the crystalline form A of Compound 1 free base has very low or nearly no hygroscopicity.

In some embodiments, the crystalline form A of Compound 1 free base can be prepared by a method that includes the following step: mixing a base (e.g., an alkali hydroxide such as sodium hydroxide or potassium hydroxide) with a solution containing Compound 1 hydrochloride in water and an alcohol (e.g., ethanol or isopropanol) to obtain the crystalline form A of Compound 1 free base. In some embodiments, the mixing step can be performed at an elevated temperature (e.g., 60-70° C.). In some embodiments, when no crystal is formed after the mixing a base with the Compound 1 hydrochloride solution, the method above can further include adding crystal seeds to the mixture to induce crystallization. In some embodiments, the method can further include cooling the mixture to a suitable temperature (e.g., −5-5° C.) after the mixing step to facilitate crystallization.

### Crystalline Form B of Compound 1 Free Base

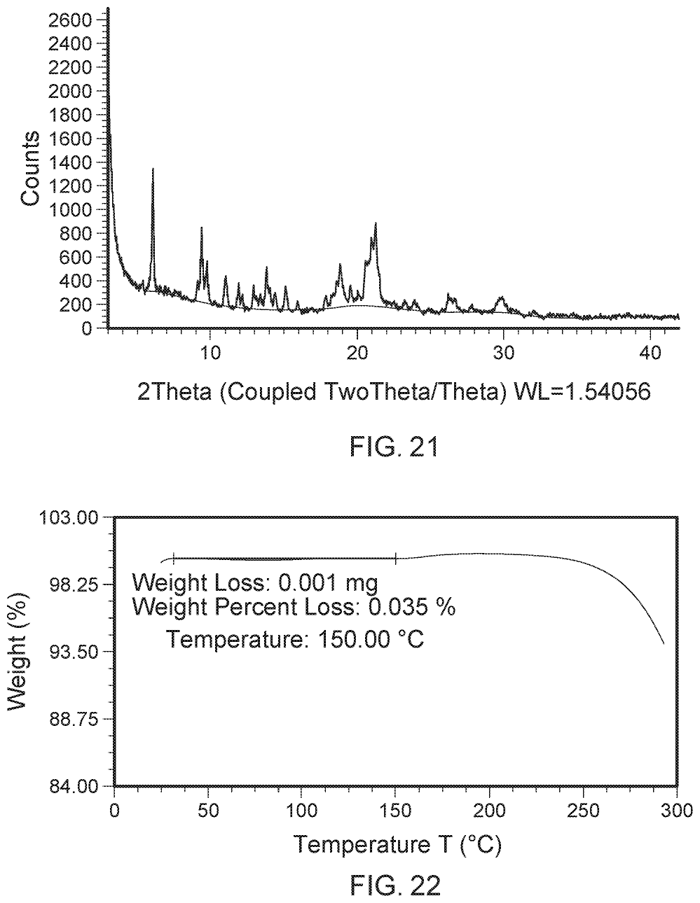

In some embodiments, this disclosure features a crystalline form B of Compound 1 free base. In some embodiments, the crystalline form B of Compound 1 free base exhibits an XRPD pattern that includes at least one (e.g., two or three) diffraction peak having a diffraction angle 2θ selected from the group consisting of 6.1±0.2°, 9.4±0.2°, and 21.3±0.2° obtained by using CuKα radiation. In some embodiments, the XRPD pattern of the crystalline form B of Compound 1 free base further includes at least one (e.g., two or three) diffraction peak having a diffraction angle 2θ selected from the group consisting of 13.8±0.2°, 18.8±0.2°, and 20.7±0.2° obtained by using CuKα radiation. In some embodiments, the XRPD pattern of the crystalline form B of Compound 1 free base further includes at least one (e.g., two or three) diffraction peak having a diffraction angle 2θ selected from the group consisting of 9.7±0.2°, 11.0±0.2°, and 11.9±0.2° obtained by using CuKα radiation. In some embodiments, the XRPD pattern of the crystalline form B of Compound 1 free base includes at least one (e.g., 2, 3, 4, 5, 6, 7, 9, 10, 11, 12, 13, 14, 15, or all) of the diffraction peaks in Table 11 below. In some embodiments, the XRPD pattern of the crystalline form B of Compound 1 free base is substantially as depicted in FIG. 21. It is believed that the crystalline form B of Compound 1 free base is an anhydrate.

In some embodiments, the crystalline form B of Compound 1 free base can be prepared by a method that includes the following steps: (1) dispersing a crystalline form A of Compound 1 free base in dichloromethane to form a dispersion (i.e., the crystal is not completely dissolved in dichloromethane); and (2) stirring the dispersion at a temperature from about 45° C. to about 55° C. (e.g., about 50° C.) to obtain the crystalline form B of Compound 1 free base. In some embodiments, the stirring step can be performed for at least 3 days (e.g., at least 7 days) and/or at most 10 days. In some embodiments, the above method can further include filtering the dispersion to obtain the crystalline form B of Compound 1 free base.

### Crystalline Form C of Compound 1 Free Base

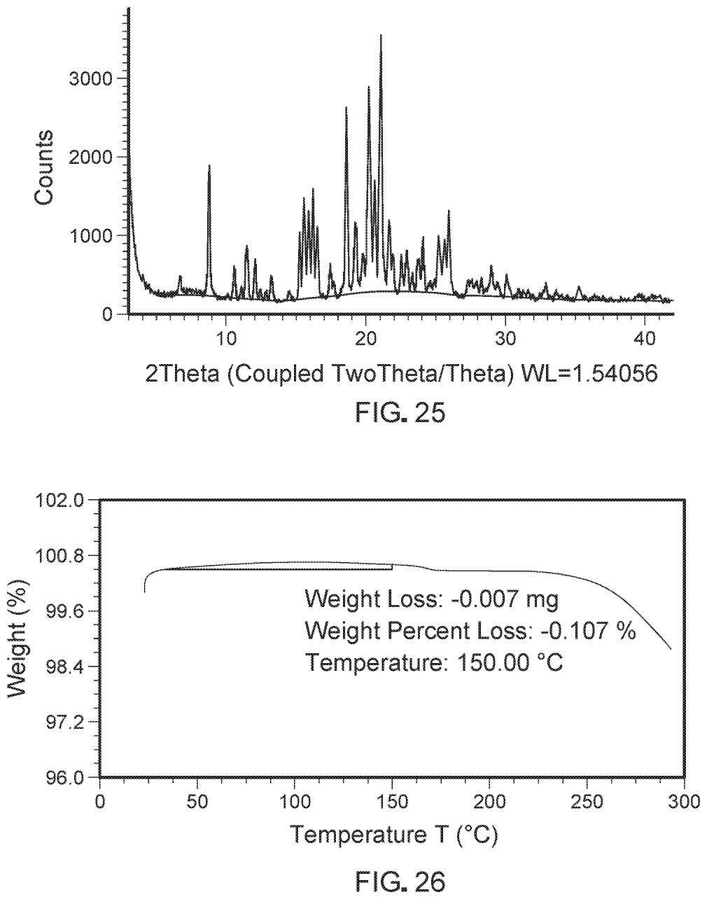

In some embodiments, this disclosure features a crystalline form C of Compound 1 free base. In some embodiments, the crystalline form C of Compound 1 free base exhibits an XRPD pattern that includes at least one (e.g., two or three) diffraction peak having a diffraction angle 2θ selected from the group consisting of 18.6±0.2°, 20.2±0.2°, and 21.1±0.2° obtained by using CuKα radiation. In some embodiments, the XRPD pattern of the crystalline form C of Compound 1 free base further includes at least one (e.g., two or three) diffraction peak having a diffraction angle 2θ selected from the group consisting of 8.8±0.2°, 16.2±0.2°, and 20.6±0.2° obtained by using CuKα radiation. In some embodiments, the XRPD pattern of the crystalline form C of Compound 1 free base further includes at least one (e.g., two or three) diffraction peak having a diffraction angle 2θ selected from the group consisting of 15.6±0.2°, 15.9±0.2°, and 25.9±0.2° obtained by using CuKα radiation. In some embodiments, the XRPD pattern of the crystalline form C of Compound 1 free base includes at least one (e.g., 2, 3, 4, 5, 6, 7, 9, 10, 11, 12, 13, 14, 15, or all) of the diffraction peaks in Table 12 below. In some embodiments, the XRPD pattern of the crystalline form C of Compound 1 free base is substantially as depicted in FIG. 25. It is believed that the crystalline form C of Compound 1 free base is an anhydrate. In general, the crystalline form C of Compound 1 free base has superior physical stability.

In some embodiments, the crystalline form C of Compound 1 free base can be prepared by a method that includes the following steps: (1) dissolving a crystalline form A of Compound 1 free base in dichloromethane to form a solution (i.e., the crystal is completely dissolved in dichloromethane); and (2) removing dichloromethane by evaporation to obtain the crystalline form C of Compound 1 free base. In some embodiments, the evaporation can be performed at an elevated temperature, such as from about 45° C. to about 55° C. (e.g., about 50° C.).

### Crystalline Form D of Compound 1 Free Base

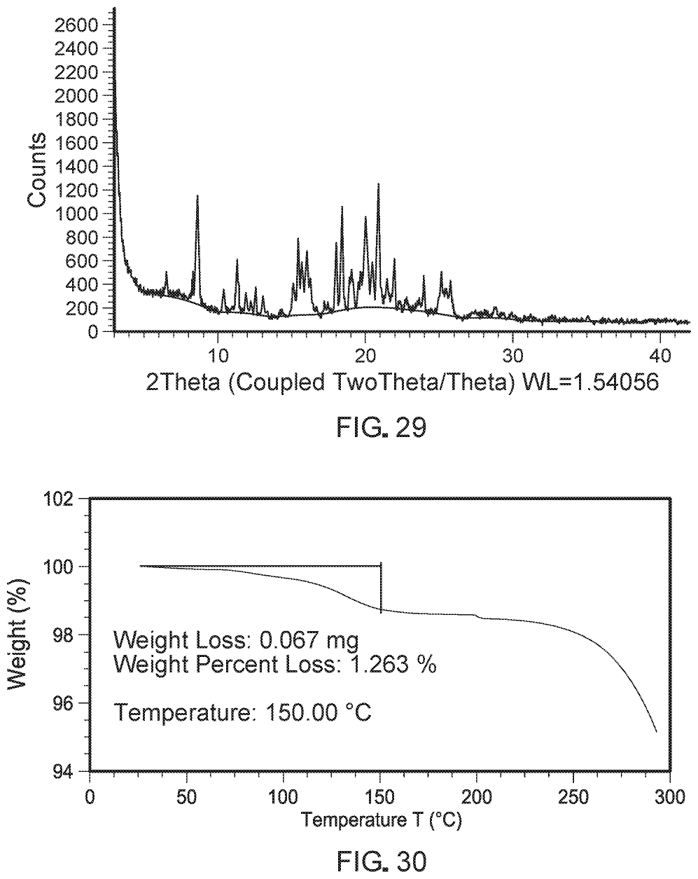

In some embodiments, this disclosure features a crystalline form D of Compound 1 free base. In some embodiments, the crystalline form D of Compound 1 free base exhibits an XRPD pattern that includes at least one (e.g., two or three) diffraction peak having a diffraction angle 2θ selected from the group consisting of 8.6±0.2°, 18.4±0.2°, and 20.9±0.2° obtained by using CuKα radiation. In some embodiments, the XRPD pattern of the crystalline form D of Compound 1 free base further includes at least one (e.g., two or three) diffraction peak having a diffraction angle 2θ selected from the group consisting of 15.5±0.2°, 18.0±0.2°, and 20.1±0.2° obtained by using CuKα radiation. In some embodiments, the XRPD pattern of the crystalline form D of Compound 1 free base further includes at least one (e.g., two or three) diffraction peak having a diffraction angle 2θ selected from the group consisting of 11.3±0.2°, 15.7±0.2°, and 16.0±0.2° obtained by using CuKα radiation. In some embodiments, the XRPD pattern of the crystalline form D of Compound 1 free base includes at least one (e.g., 2, 3, 4, 5, 6, 7, 9, 10, 11, 12, 13, 14, 15, or all) of the diffraction peaks in Table 13 below. In some embodiments, the XRPD pattern of the crystalline form D of Compound 1 free base is substantially as depicted in FIG. 29. It is believed that the crystalline form D of Compound 1 free base is a solvate/hydrate mixture.

In some embodiments, the crystalline form D of Compound 1 free base can be prepared by a method that includes the following steps: (1) dissolving a crystalline form A of Compound 1 free base in methanol to form a solution; (2) adding methyl tert-butyl ether to the solution; and (3) removing methanol and methyl tert-butyl ether by evaporation to obtain the crystalline form D of Compound 1 free base. In some embodiments, the evaporation can be performed by using a rotary evaporator at an elevated temperature (e.g., about 40-45° C.).

### Pharmaceutical Compositions

This disclosure also features pharmaceutical compositions containing a therapeutically effective amount of at least one (e.g., two or more) of the crystalline forms of Compound 1 or a salt thereof (e.g., a pharmaceutically acceptable salt thereof) as an active ingredient, as well as at least one pharmaceutically acceptable carrier (e.g., adjuvant or diluent). Examples of pharmaceutically acceptable salts include acid addition salts, e.g., salts formed by reaction between Compound 1 and hydrohalogen acids (such as hydrochloric acid or hydrobromic acid), mineral acids (such as sulfuric acid, phosphoric acid and nitric acid), and aliphatic, alicyclic, aromatic or heterocyclic sulfonic or carboxylic acids (such as formic acid, acetic acid, propionic acid, succinic acid, adipic acid, glycolic acid, lactic acid, malic acid, tartaric acid, citric acid, benzoic acid, ascorbic acid, maleic acid, hydroxymaleic acid, pyruvic acid, p-hydroxybenzoic acid, embonic acid, methanesulphonic acid, ethanesulphonic acid, hydroxyethanesulphonic acid, halobenzenesulphonic acid, trifluoroacetic acid, trifluoromethanesulphonic acid, toluenesulphonic acid, and naphthalenesulphonic acid).

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical carriers for delivery of a crystalline form of Compound 1 or its salt described herein. Examples of other carriers include colloidal silicon oxide, magnesium stearate, cellulose, sodium lauryl sulfate, and D&C Yellow #10.

The pharmaceutical composition described herein can optionally include at least one further additive selected from a disintegrating agent, binder, lubricant, flavoring agent, preservative, colorant and any mixture thereof. Examples of such and other additives can be found in “Handbook of Pharmaceutical Excipients”; Ed. A. H. Kibbe, 3rd Ed., American Pharmaceutical Association, USA and Pharmaceutical Press UK, 2000.

The pharmaceutical composition described herein can be adapted for parenteral, oral, topical, nasal, rectal, buccal, or sublingual administration or for administration via the respiratory tract, e.g., in the form of an aerosol or an air-suspended fine powder. The term “parenteral” as used herein refers to subcutaneous, intracutaneous, intravenous, intramuscular, intraarticular, intraarterial, intrasynovial, intrasternal, intrathecal, intralesional, intraperitoneal, intraocular, intra-aural, or intracranial injection, as well as any suitable infusion technique. In some embodiments, the composition can be in the form of tablets, capsules, powders, microparticles, granules, syrups, suspensions, solutions, nasal spray, transdermal patches, injectable solutions, or suppositories.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art.

A composition having one or more crystalline forms of Compound 1 or its salt can also be administered in the form of suppositories for rectal administration.

### Methods of Treatment

In addition, this disclosure features a method of using a crystalline form of Compound 1 or its salt as outlined above for treating cancer or for the manufacture of a medicament for such a treatment. The method can include administering to a subject (e.g., a patient) in need thereof the pharmaceutical composition described herein in an amount therapeutically effective to treat the cancer (e.g., solid tumor). In some embodiments, the cancer can have a ROS1 fusion mutation (e.g., in a ROS1-positive cancer) or a NTRK fusion mutation such as a fusion mutation in NTRK1, NTRK2, and/or NTRK3 (e.g., in an NTRK-positive cancer). In some embodiments, the cancer can have a detectable increase in the expression level of ROS1 gene and/or a detectable increase in the expression level of NTRK gene. In some embodiments, the cancer can have a detectable expression of ROS1 fusion gene and/or a detectable expression of NTRK fusion gene. In some embodiments, the cancer can be treatable by inhibition of ROS1 kinase enzyme activity and/or inhibition of NTRK kinase enzyme activity. Specific examples of such cancers (e.g., malignant cancers) include lung cancer (e.g., non-small cell lung cancer), thyroid cancer, colorectal cancer, leukemia, lymphoma, multiple myeloma, brain tumor, head and neck cancer, esophageal cancer, gastric cancer, appendix cancer, anus cancer, gallbladder cancer, bile duct cancer, pancreatic cancer, gastrointestinal stromal tumor, liver cancer, mesothelioma, kidney cancer, prostate cancer, neuroendocrine tumor, melanoma, breast cancer, uterine body cancer, uterine cervical cancer, ovary cancer, osteosarcoma, soft tissue sarcoma, Kaposi's sarcoma, myosarcoma, urinary bladder cancer, or testicular cancer, glioblastoma, and non-Hodgkin lymphoma (e.g., anaplastic large cell lymphoma). In some embodiments, the cancer that can be treated by the crystalline forms described herein can be a systemic cancer, relapsed cancer, or refractory cancer. “A therapeutically effective amount” refers to the amount of the pharmaceutical composition that is required to confer a therapeutic effect on the treated subject.

As used herein, the terms “treatment,” “treat,” and “treating” refer to reversing, alleviating, delaying the onset of, or inhibiting the progress of, a cancer or one or more symptoms thereof, as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent or delay their recurrence.

The typical dosage of the crystalline forms of Compound 1 or its salts described herein can vary within a wide range and will depend on various factors, such as the types of diseases treated, the individual needs of each patient, route of administration, excipient usage, and the possibility of co-usage with other therapeutic treatment. Exemplary daily dosages can be at least about 0.1 mg (e.g., at least about 0.5 mg, at least about 1 mg, at least about 5 mg, at least about 10 mg, at least about 15 mg, at least about 20 mg, at least about 50 mg, or at least about 100 mg) and/or at most about 800 mg (e.g., at most about 700 mg, at most about 600 mg, at most about 500 mg, at most about 400 mg, at most about 300 mg, at most about 200 mg, at most about 100 mg, at most about 75 mg, at most about 50 mg, at most about 20 mg, or at most about 15 mg) of a crystalline form of Compound 1 or a salt thereof. The skilled person or physician may consider relevant variations to this dosage range and practical implementations to accommodate the situation at hand.

In some embodiments, the pharmaceutical composition described herein can be administered once daily. In some embodiments, the pharmaceutical composition can be administered more than once daily (e.g., twice daily, three times daily, or four times daily).

The present disclosure also features a method of inhibiting ROS1 and/or NTRK kinase enzyme activity in a cell (e.g., in a patient body or in a tissue sample obtained from a patient). The method includes contacting the cell with a crystalline form of Compound 1 or its salts described herein in an amount sufficient to inhibit ROS1 and/or NTRK kinase enzyme activity in the cell.

The contents of all publications cited herein (e.g., patents, patent application publications, and articles) are hereby incorporated by reference in their entirety.

The following examples are illustrative and not intended to be limiting.

### EXAMPLES

### Instruments and Analytical Methods

The following instruments and measurement methods were used in the Examples described below:

### X-Ray Powder Diffraction

X-ray powder diffraction (XRPD) patterns were obtained by using a Bruker D8 Focus X-ray powder diffractometer using CuKα radiation at 1.54056 Å at a voltage of 40 kV and at a current of 40 mA. The XRPD analysis was performed through measurement at an angle 2θ in a scan range of 3° to 420 with a scan step of 0.02° and a scan time of 0.2 second for each step. During measurement, a suitable amount of a sample was placed on a sample plate of the diffractometer and was flattened by using a spatula or a glass slide.

### Thermogravimetric Analysis

Thermogravimetric Analysis (TGA) was performed by using TA Instruments TGA Discovery 550. A sample was placed in an aluminum pan and was weighed by the instrument. The sample was evaluated under N₂ (50 ml/min) by using a linear heat ramp of 10° C./min to the predetermined temperature.

### Differential Scanning Calorimetry

Differential Scanning Calorimetry (DSC) was performed by using TA Instruments Discovery DSC 25. A sample was weighed and placed in an aluminum pan with a cover. The sample was evaluated under N₂ (50 ml/min) by using a linear heat ramp of 10° C./min to the predetermined temperature.

### Dynamic Vapor Sorption

Dynamic Vapor Sorption (DVS) was performed by using Intrinsic DVS (Surface Measurement Systems, UK). A sample was weighed in an amount of 20-30 mg and was placed in a sample chamber. The measurement was performed at DMDT mode when the temperature of the sample chamber was maintained at 25±1° C.

### Polarized Light Microscopy

Polarized light microscopy (PLM) was performed by using a DM750P polarized light microscope. The magnification range was adjusted to obtain the morphology and microstructure of the sample.

### High Pressure Liquid Chromatography

High pressure liquid chromatography (HPLC) was performed by using the instrument and parameters summarized in Table 1.

TABLE 1

| HPLC | Agilent 1260 (DAD detector) | |
|---|---|---|
| Column | Waters ACQUITY BEH C18, 2.1 × 75 mm, 1.7 μm | |
| Mobile Phase | A: 10 mM Phosphate buffer(Ph = 7.2):ACN:MeOH = 34:3:3(v/v/v) | |
| | B: 10 mM Phosphate buffer(Ph = 7.2):ACN:MeOH = 6:17:17(v/v/v) | |
| Gradient | Time (min) | % B |
| | 0.0 | 14 |
| | 0.5 | 14 |
| | 15.5 | 86 |
| | 15.51 | 14 |
| | 19.0 | 14 |

| | |
|---|---|
| Speed | 0.5 mL/min |
| Injection Volume | 2 μL |
| Column Temperature | 50° C. |
| Sample Plate | 10° C. |
| Temperature | |
| Diluent | Acetonitrile:10 mM NaOH in H₂O = 50:50 (v/v) |

### Example 1: Preparation and Characterization of Crystalline Form a of Compound 1 Adipate

A suitable amount of an amorphous form of Compound 1 adipate was dissolved in 0.5 mL of a solvent to form a solution. An anti-solvent was added to the solution thus obtained to obtain a solid, which was confirmed to be the crystalline form A of Compound 1 adipate. If no solid was formed after addition of the anti-solvent, the solvent and anti-solvent were removed by blow drying to obtain a solid. A number of solvents and anti-solvents were used in the above experiments and are summarized in Table 2 below.

TABLE 2

| | | Concentration | Anti- | | |
|---|---|---|---|---|---|
| No. | Solvent | (mg/mL) | Solvent | Volume (mL) | XRPD |
| 1 | EtOH | 20.5 | THF | 5 | Amorphous |
| 2 | | | ACN | 5 | Form A |
| 3 | | | water | 5 | Form A |
| 4 | | | acetone | 5 | Form C |
| 5 | | | EA | 2 | Form A |
| 6 | | | heptane | 5 | Form A |
| 7 | | | MTBE | 5 | Form A |
| 8 | | | toluene | 5 | Form A |
| 9 | IPA | 21.46 | THF | 5 | Amorphous |
| 10 | | | ACN | 5 | Form A |
| 11 | | | water | 5 | Form A |
| 12 | | | acetone | 5 | Form A |
| 13 | | | EA | 5 | Form A |
| 14 | | | heptane | 1 | Form A |
| 15 | | | MTBE | 3.5 | Form A |
| 16 | | | toluene | 5 | Form A |
| 17 | DMSO | 60.82 | THF | 5 | N/A |
| 18 | | | ACN | 5 | Form A |
| 19 | | | water | 5 | Form A |
| 20 | | | acetone | 5 | Form A |
| 21 | | | EA | 5 | Form A |
| 22 | | | toluene | 5 | Form A |
| 23 | | | MTBE | 1 | Form A |
| 24 | | | DCM | 5 | Form A |
| 25 | DMF | 60.87 | THF | 5 | Form A |
| 26 | | | ACN | 1.5 | Form A |
| 27 | | | water | 5 | Form A |
| 28 | | | acetone | 5 | Form A |
| 29 | | | EA | 5 | Form A |
| 30 | | | toluene | 3 | Form A |
| 31 | | | MTBE | 1.5 | Form A |
| 32 | | | DCM | 5 | Form A |
| 33 | DMAc | 59.88 | THF | 5 | Form A |
| 34 | | | ACN | 4 | Form A |
| 35 | | | water | 5 | Form A |
| 36 | | | acetone | 5 | Form D |
| 37 | | | EA | 5 | Form A |
| 38 | | | toluene | 5 | Form A |
| 39 | | | MTBE | 2 | Form A |
| 40 | | | DCM | 5 | Form A |

As shown in Table 2, most solvent/anti-solvent combinations formed the crystalline form A of Compound 1 adipate except that certain solvent/anti-solvent combinations formed an amorphous form, or the crystalline form C or D of Compound 1 adipate.

The NMR data for the crystalline form A of Compound 1 adipate are as follows: 1H NMR (500 MHz, DMSO) δ 1.13-1.14 (d, J=5.0 Hz, 3H), 1.47-1.48 (d, J=5.0 Hz, 7H), 2.15-2.18 (t, J=5.0 Hz, J=10.0 Hz, 4H), 3.25-3.29 (m, 1H), 3.79-3.83 (m, 2H), 4.80-4.85 (m, 1H), 6.76-6.77 (d, J=5.0 Hz, 1H), 6.92-6.94 (d, J=10.0 Hz, 2H), 7.01-7.05 (t, J=10.0 Hz, 1H), 7.23-7.28 (m, 2H), 7.37-7.42 (m, 1H), 7.64-7.65 (d, J=5.0 Hz, 1H), 7.72-7.76 (t, J=10.0 Hz, 4H).

The IR data for the crystalline form A of Compound 1 adipate are as follows: IR (cm⁻¹): 1701, 1628, 1612, 1586, 1463, 1333, 1246, 1110, 829, 821.

A representative XRPD pattern obtained from the crystalline form A of Compound 1 adipate is shown in FIG. 1 and the XRPD data are listed in Table 3 below.

TABLE 3

| Index | Angle | d Value | Rel. Intensity |
|---|---|---|---|
| 1 | 5.806° | 15.21020 Å | 95.6% |
| 2 | 9.660° | 9.14867 Å | 4.9% |
| 3 | 10.181° | 8.68144 Å | 1.3% |
| 4 | 11.722° | 7.54335 Å | 10.6% |
| 5 | 13.087° | 6.75957 Å | 1.9% |
| 6 | 13.350° | 6.62683 Å | 5.3% |
| 7 | 13.728° | 6.44502 Å | 11.0% |
| 8 | 14.823° | 5.97123 Å | 3.0% |
| 9 | 15.978° | 5.54225 Å | 1.2% |
| 10 | 16.541° | 5.35487 Å | 1.6% |
| 11 | 17.201° | 5.15100 Å | 4.3% |
| 12 | 17.423° | 5.08582 Å | 10.2% |
| 13 | 18.468° | 4.80031 Å | 15.5% |
| 14 | 18.968° | 4.67477 Å | 7.0% |
| 15 | 19.359° | 4.58123 Å | 16.9% |
| 16 | 19.731° | 4.49580 Å | 7.0% |
| 17 | 20.438° | 4.34180 Å | 5.3% |
| 18 | 20.657° | 4.29614 Å | 14.9% |
| 19 | 21.097° | 4.20756 Å | 47.7% |
| 20 | 21.456° | 4.13810 Å | 2.4% |
| 21 | 21.905° | 4.05415 Å | 1.3% |
| 22 | 22.431° | 3.96033 Å | 7.8% |
| 23 | 23.294° | 3.81558 Å | 100.0% |
| 24 | 23.589° | 3.76847 Å | 6.4% |
| 25 | 24.501° | 3.63020 Å | 1.7% |
| 26 | 25.330° | 3.51327 Å | 1.2% |
| 27 | 26.471° | 3.36435 Å | 3.6% |
| 28 | 26.765° | 3.32806 Å | 3.8% |
| 29 | 27.849° | 3.20097 Å | 4.1% |
| 30 | 28.099° | 3.17304 Å | 2.0% |
| 31 | 28.775° | 3.10001 Å | 5.4% |
| 32 | 29.233° | 3.05244 Å | 27.3% |
| 33 | 29.693° | 3.00618 Å | 2.5% |
| 34 | 30.264° | 2.95076 Å | 3.4% |
| 35 | 30.683° | 2.91143 Å | 1.0% |
| 36 | 32.106° | 2.78560 Å | 2.0% |
| 37 | 32.450° | 2.75683 Å | 1.9% |
| 38 | 33.578° | 2.66673 Å | 2.3% |
| 39 | 35.245° | 2.54430 Å | 3.2% |
| 40 | 36.835° | 2.43809 Å | 0.7% |
| 41 | 37.317° | 2.40771 Å | 0.7% |
| 42 | 38.620° | 2.32941 Å | 0.7% |
| 43 | 39.840° | 2.26083 Å | 1.2% |
| 44 | 41.360° | 2.18116 Å | 0.7% |

The TGA curve obtained from the crystalline form A of Compound 1 adipate is shown in FIG. 2. As shown in FIG. 2, the crystalline form A of Compound 1 adipate exhibited a weight loss of 0.072% when heated to 150° C.

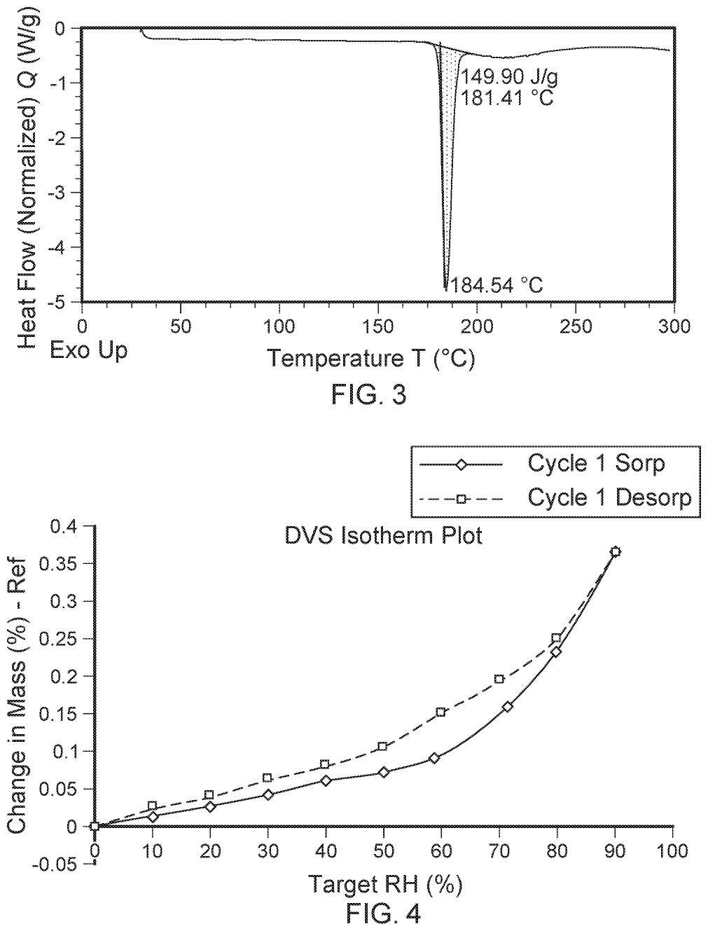

The DSC curve obtained from the crystalline form A of Compound 1 adipate is shown in FIG. 3. As shown in FIG. 3, the crystalline form A of Compound 1 adipate exhibited an endothermic peak at 181.41° C.

The DVS curve obtained from the crystalline form A of Compound 1 adipate is shown in FIG. 4. As shown in FIG. 4, the crystalline form A of Compound 1 adipate exhibited a weight increase of 0.235% at 80% RH, which showed a low hygroscopicity. In addition, the XRPD shows that the crystalline form of the sample before and after the DVS test did not change.

The above results suggest that the crystalline form A of Compound 1 adipate is an anhydrate.

### Solubility

The solubility of the crystalline form A of Compound 1 adipate was evaluated by using the following procedures. Specifically, 50 mg of the crystalline form A of Compound 1 adipate was mixed with 2 mL of the following media: (1) a KCl/HCl buffer solution with a pH of 1.0, (2) a potassium hydrogen phthalate buffer solution with a pH of 3.0, (3) a sodium acetate trihydrate buffer solution with a pH of 4.5, (4) a potassium dihydrogen phosphate buffer solution with a pH of 6.0, (5) a potassium dihydrogen phosphate buffer solution with a pH of 7.5, (6) SGF (simulated gastric fluid), (7) FaSSIF (fasted state simulated intestinal fluids, pH=6.5), (8) FeSSIF (fed state simulated intestinal fluids, pH=5.0), and (9) water. Each mixture was then stirred in a water bath at 37° C. After equilibrated for 2 hours and 24 hours, concentrations (mg/mL) of the crystal in the mixture were measured by HPLC and the undissolved solid was evaluated by XRPD. The results are summarized in Table 4 below.

TABLE 4

| | | pH after 24 | | Solubility at 2-hour | Solubility at 24 hour |
|---|---|---|---|---|---|
| No. | Medium | hours | XRPD | (mg/mL) | (mg/mL) |
| 1 | pH 1.0 | 3.746 | changed | 20.431 | 17.820 |
| 2 | pH 3.0 | 4.418 | changed | 1.348 | 0.852 |
| 3 | pH 4.5 | 4.541 | Form A | 2.839 | 2.885 |
| 4 | pH 6.0 | 5.055 | Form A | 2.337 | 1.703 |
| 5 | pH 7.5 | 5.570 | changed | 0.597 | 0.419 |
| 6 | SGF | 5.281 | changed | 15.611 | 15.990 |
| 7 | FaSSIF | 5.008 | changed | 0.331 | 0.225 |
| 8 | FeSSIF | 4.205 | Form A | 0.493 | 0.143 |
| 9 | Water | 5.115 | Form A | 1.606 | 1.413 |

As shown in Table 4, the crystalline form A of Compound 1 adipate exhibited superior solubility in the buffer with a pH of 1.0 and SGF.

### Stability

A predetermined amount of the crystalline form A of Compound 1 adipate was stored under the following conditions: (1) open to air under 25° C./60% RH for one or two weeks, (2) open to air under 40° C./75% RH for one or two weeks, (3) open to air under 80° C. for one day, and (4) exposed to light for 10 days. Crystalline form and chemical impurity were checked by XRPD and HPLC, respectively. The results are summarized in Table 5.

TABLE 5

| No. | Conditions | Time | | XRPD | Purity (%) |
|---|---|---|---|---|---|
| 1 | starting material | | | Form A | 99.86 |
| 2 | 25° C./60% RH | 1 | week | Form A | 99.81 |
| 3 | | 2 | weeks | Form A | 99.79 |
| 4 | 40° C./75% RH | 1 | week | Form A | 99.84 |
| 5 | | 2 | weeks | Form A | 99.81 |
| 6 | 80° C. | 1 | day | Form A | 99.68 |
| 7 | Exposure to light (1.2 million lux h) | 10 | days | Form A | 99.79 |
| 8 | Exposure to light (1.2 million lux h) | 10 | days | Form A | 99.81 |

In Table 5, Sample No. 1 was a crystalline form A starting material before any test; Samples Nos. 2 and 3 were subject to condition (1) described above; Samples Nos. 4 and 5 were subject to condition (2) described above; Samples No. 6 was subject to condition (3) described above; and Samples Nos. 7 and 8 were subject to condition (4) described above where Sample No. 7 was not covered and Sample No. 8 was completely covered by a tin foil as a comparison. As shown in Table 5, the crystalline form A of Compound 1 adipate exhibited superior physical and chemical stability under stress conditions.

The accelerated stability test of the crystalline form A of Compound 1 adipate was performed by storing a sample at 40° C.±2° C./75±500 RH for six months. The results are summarized in Table 6 below.

TABLE 6
Storage condition: 40° C. ± 2° C./75 ± 5% RH

| Testing item | | Acceptance criteria | 0 M | 1 M | 2 M | 3 M | 6 M |
|---|---|---|---|---|---|---|---|
| Appearance | | White to yellowish | White | White | White | White | White |
| | | powder | powder | powder | powder | powder | powder |
| Water content (KF) | | Report result | 0.2% | 0.1% | 0.1% | 0.1% | 0.1% |
| Related | deF-6051 | ≤0.50% | N.D. | N.D. | N.D. | N.D. | N.D. |
| substances | Unspecified | ≤0.10% | RRT1.48: | <0.05% | <0.05% | <0.05% | <0.05% |
| (UPLC) | individual | | 0.05% | | | | |
| | impurity | | | | | | |
| | Total | ≤1.00% | 0.05% | <0.05% | <0.05% | <0.05% | <0.05% |
| | impurities | | | | | | |
| Stereoisomer | SR-form | ≤0.10% | N.D. | N.D. | N.D. | N.D. | N.D. |
| (HPLC) | RS-form | ≤0.10% | N.D. | N.D. | N.D. | N.D. | N.D. |
| | SS-form | ≤0.10% | N.D. | N.D. | N.D. | N.D. | N.D. |
| Assay | DS-6051a | 97.0- | 99.5% | 100.1% | 99.6% | 99.5% | 99.0% |
| | (HPLC)/ | 102.0% | | | | | |
| | (anhydrous | | | | | | |
| | and solvent | | | | | | |
| | free basis) | | | | | | |
| | Adipic acid | 25.2- | 26.2% | 26.5% | 26.4% | 26.2% | 26.2% |
| | (potentiometric | 27.8% | | | | | |
| | titration) | | | | | | |
| Crystal form (XRPD) | | The crystal form of | conform | conform | conform | conform | conform |
| | | the sample is | | | | | |
| | | consistent with that | | | | | |
| | | of reference standard | | | | | |
| Microbial | Total | ≤10³ CFU/g | <100 CFU/g | / | / | / | <100 CFU/g |
| limit | aerobic | | | | | | |
| | microbial | | | | | | |
| | count | | | | | | |
| | total yeasts | ≤10² CFU/g | <100 CFU/g | | | | <100 CFU/g |
| | and molds | | | | | | |
| | count | | | | | | |
| | Escherichia | Absence per 1 gram | Absence | | | | Absence |
| | coli | | | | | | |

As shown in Table 6, the crystalline form A of Compound 1 adipate exhibited superior stability under the accelerated test conditions above.

The long-term stability test of the crystalline form A of Compound 1 adipate was performed by storing a sample at 25°±2° C./60±50 RH for three years. The results are summarized in Table 7 below.

TABLE 7
Storage condition: 25° C. ± 2° C./60 ± 5% RH

| Testing | Acceptance | | | | | | | | |
|---|---|---|---|---|---|---|---|---|---|
| item | criteria | 0 M | 3 M | 6 M | 9 M | 12 M | 18 M | 24 M | 36 M |
| Appearance | White to | White | White | White | White | White | White | White | White |
| | yellowish | powder | powder | powder | powder | powder | powder | powder | powder |
| | powder | | | | | | | | |
| deF-6051 | ≤1.00% | 0.46 | 0.46 | 0.45 | 0.46 | 0.46 | 0.46 | 0.45 | 0.44 |
| Individual | ≤0.30% | Maximum: | Maximum: | Maximum: | Maximum: | Maximum: | Maximum: | Maximum: | Maximum: |
| unspecified | | 0.09 | 0.09 | 0.09 | 0.09 | 0.09 | 0.09 | 0.09 | 0.09 |
| impurity | | | | | | | | | |
| Total | ≤3.00% | 0.61 | 0.61 | 0.54 | 0.60 | 0.55 | 0.55 | 0.54 | 0.53 |
| impurities | | | | | | | | | |
| Water | Report | 0.07 | 0.08 | 0.11 | 0.08 | 0.09 | 0.06 | 0.07 | 0.06 |
| content | result (%) | | | | | | | | |
| Assay | 97.0-102.0% | 100.0 | 100.0 | 100.0 | 99.9 | 100.2 | 100.2 | 99.7 | 100.0 |
| (anhydrous | | | | | | | | | |
| and solvent | | | | | | | | | |
| free basis) | | | | | | | | | |

As shown in Table 7, the crystalline form A of Compound 1 adipate exhibited superior stability under the long-term test conditions above.

### Example 2: Preparation and Characterization of Crystalline Form B of Compound 1 Adipate

12 mg of an amorphous form of Compound 1 adipate was placed in a 30 mL beaker. 1 mL of a mixture of dichloromethane and methanol at an 1:1 volume ratio was added to the beaker to dissolve the amorphous form of Compound 1 adipate. The solvent was allowed to evaporate slowly at room temperature. After the solvent was completely evaporated, a solid was obtained and was confirmed to be the crystalline form B of Compound 1 adipate.

The NMR data for the crystalline form B of Compound 1 adipate are as follows: 1H NMR (500 MHz, DMSO) δ 1.15 (br, 3H), 1.48 (br, 7H), 2.17 (br, 4H), 3.28 (br, 1H), 3.85 (br, 2H), 4.823 (br, 1H), 6.77 (br, 1H), 6.94 (br, 2H), 7.03 (br, 1H), 7.26 (br, 2H), 7.40 (br, 1H), 7.65 (br, 1H), 7.74 (br, 4H).

The IR data for the crystalline form B of Compound 1 adipate are as follows: IR (cm⁻¹): 3274, 3058, 2972, 2937, 2868, 1700, 1612, 1574, 1333, 1245, 1111, 829, 821.

A representative XRPD pattern obtained from the crystalline form B of Compound 1 adipate is shown in FIG. 5 and the XRPD data are listed in Table 8 below.

TABLE 8

| Index | Angle | d Value | Rel. Intensity |
|---|---|---|---|
| 1 | 5.238° | 16.85764 Å | 100.0% |
| 2 | 7.176° | 12.30905 Å | 84.0% |
| 3 | 12.528° | 7.05989 Å | 5.7% |
| 4 | 14.536° | 6.08883 Å | 8.6% |
| 5 | 15.335° | 5.77330 Å | 4.6% |
| 6 | 17.335° | 5.11123 Å | 13.1% |
| 7 | 19.397° | 4.57236 Å | 6.7% |
| 8 | 20.546° | 4.31917 Å | 10.8% |
| 9 | 20.902° | 4.24648 Å | 17.1% |
| 10 | 22.228° | 3.99606 Å | 10.6% |
| 11 | 25.661° | 3.46871 Å | 8.7% |
| 12 | 26.216° | 3.39647 Å | 7.2% |

The TGA curve obtained from the crystalline form B of Compound 1 adipate is shown in FIG. 6. As shown in FIG. 6, the crystalline form B of Compound 1 adipate exhibited a weight loss of about 9.7% when heated to 150° C.

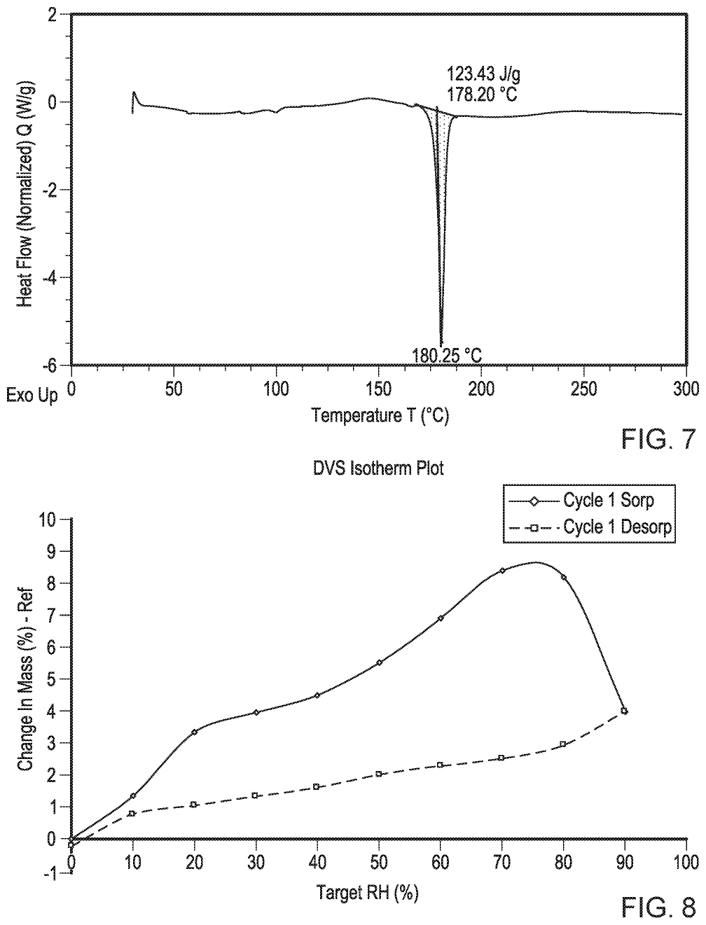

The DSC curve obtained from the crystalline form B of Compound 1 adipate is shown in FIG. 7. As shown in FIG. 7, the crystalline form B of Compound 1 adipate exhibited an endothermic peak at 178.20° C.

The DVS curve obtained from the crystalline form B of Compound 1 adipate is shown in FIG. 8. As shown in FIG. 8, the crystalline form B of Compound 1 adipate exhibited a weight increase of about 8.2% at 80% RH, which suggests that it has hygroscopicity.

After a sample was heated to 150° C., its XRPD shows that the crystalline form of the sample changed.

The above results suggest that the crystalline form B of Compound 1 adipate is a hydrate. In addition, it is believed that the crystalline form B of Compound 1 adipate includes about 3.6 molar water per molar Compound 1 adipate.

### Example 3: Preparation and Characterization of Crystalline Form C of Compound 1 Adipate

10 mg of an amorphous form of Compound 1 adipate was placed in a 30 mL beaker. 0.5 mL of ethanol was added to the beaker to dissolve the amorphous form of Compound 1 adipate. After 5 mL of acetone was added to the beaker, the solution was stirred at room temperature for one day. The solvent was then removed by blowing nitrogen to the solution to obtain a solid, which was confirmed to be the crystalline form C of Compound 1 adipate.

The NMR data for the crystalline form C of Compound 1 adipate are as follows: 1H NMR (500 MHz, DMSO) δ 1.13-1.14 (d, J=5.0 Hz, 3H), 1.47-1.48 (d, J=5.0 Hz, 7H), 2.14-2.18 (t, J=5.0 Hz, J=10.0 Hz, 4H), 3.25-3.29 (m, 1H), 3.81-3.88 (m, 2H), 4.81-4.86 (m, 1H), 6.76-6.78 (d, J=10.0 Hz, 1H), 6.93-6.94 (d, J=5.0 Hz, 2H), 7.01-7.05 (t, J=10.0 Hz, 1H), 7.23-7.28 (m, 2H), 7.37-7.42 (m, 1H), 7.64-7.65 (d, J=5.0 Hz, 1H), 7.72-7.76 (t, J=10.0 Hz, 4H).

The IR data for the crystalline form C of Compound 1 adipate are as follows: IR (cm⁻¹): 3275, 3057, 2974, 2939, 2868, 1700, 1612, 1583, 1333, 1245, 1110, 829, 821.

A representative XRPD pattern obtained from the crystalline form C of Compound 1 adipate is shown in FIG. 9 and the XRPD data are listed in Table 9 below.

TABLE 9

| Index | Angle | d Value | Rel. Intensity |
|---|---|---|---|
| 1 | 5.436° | 16.24377 Å | 13.0% |
| 2 | 5.724° | 15.42696 Å | 100.0% |
| 3 | 11.625° | 7.60566 Å | 4.9% |
| 4 | 13.592° | 6.50954 Å | 13.1% |
| 5 | 18.407° | 4.81613 Å | 9.0% |
| 6 | 19.300° | 4.59518 Å | 6.0% |
| 7 | 20.613° | 4.30522 Å | 8.6% |
| 8 | 21.041° | 4.21876 Å | 27.3% |
| 9 | 21.684° | 4.09499 Å | 6.1% |
| 10 | 22.381° | 3.96897 Å | 4.4% |
| 11 | 23.199° | 3.83089 Å | 54.0% |
| 12 | 29.158° | 3.06014 Å | 14.4% |

The TGA curve obtained from the crystalline form C of Compound 1 adipate is shown in FIG. 10. As shown in FIG. 10, the crystalline form C of Compound 1 adipate exhibited a weight loss of about 38.1% when heated to 150° C.

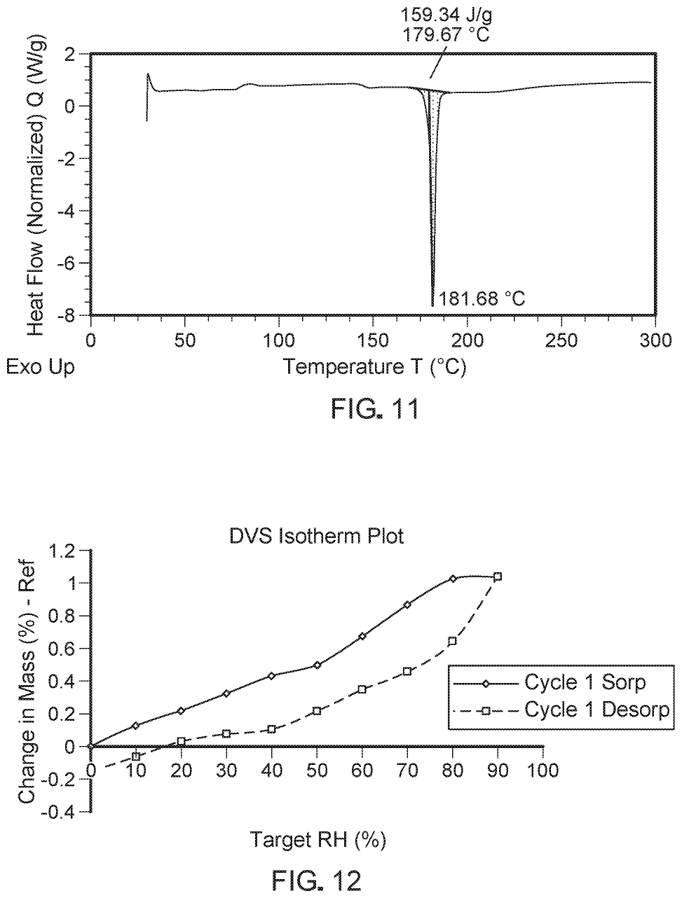

The DSC curve obtained from the crystalline form C of Compound 1 adipate is shown in FIG. 11. As shown in FIG. 11, the crystalline form C of Compound 1 adipate exhibited an endothermic peak at 179.67° C.

The DVS curve obtained from the crystalline form C of Compound 1 adipate is shown in FIG. 12. As shown in FIG. 12, the crystalline form C of Compound 1 adipate exhibited a weight increase of about 1% at 80% RH, which suggests that it has low hygroscopicity.

After a sample was heated to 160° C., its XRPD shows that the crystalline form of the sample changed.

The above results suggest that the crystalline form C of Compound 1 adipate is a hydrate. In addition, it is believed that the crystalline form C of Compound 1 adipate includes about 13 molar water per molar Compound 1 adipate.

### Example 4: Preparation and Characterization of Crystalline Form D of Compound 1 Adipate

30 mg of an amorphous form of Compound 1 adipate was placed in a 30 mL beaker. 0.5 mL of dimethylacetamide (DMAc) was added to the beaker to dissolve the amorphous form of Compound 1 adipate. After 5 mL of acetone was added to the beaker, the solution was stirred at room temperature for one day. The solvent was then removed by blowing nitrogen to the solution to obtain a solid, which was confirmed to be the crystalline form D of Compound 1 adipate.

The NMR data for the crystalline form D of Compound 1 adipate are as follows: 1H NMR (500 MHz, DMSO) δ 1.10-1.12 (d, J=10.0 Hz, 3H), 1.47-1.49 (d, J=10.0 Hz, 7H), 1.96 (s, 3H), 2.17-2.20 (t, J=5.0 Hz, J=10.0 Hz, 4H), 2.78 (s, 3H), 2.94 (s, 3H), 3.21-3.24 (m, 1H), 3.83-3.86 (m, 2H), 4.82-4.85 (m, 1H), 6.76-6.78 (d, J=10.0 Hz, 1H), 6.93-6.94 (d, J=5.0 Hz, 2H), 7.01-7.05 (t, J=10.0 Hz, 1H), 7.23-7.28 (m, 2H), 7.37-7.42 (m, 1H), 7.63-7.64 (d, J=5.0 Hz, 1H), 7.72-7.76 (t, J=10.0 Hz, 4H).

The IR data for the crystalline form D of Compound 1 adipate are as follows: IR (cm⁻¹): 2937, 2873, 1628, 1613, 1583, 1457, 1333, 1242, 1110, 829, 821.

A representative XRPD pattern obtained from the crystalline form D of Compound 1 adipate is shown in FIG. 13 and the XRPD data are listed in Table 10 below.

TABLE 10

| Index | Angle | d Value | Rel. Intensity |
|---|---|---|---|
| 1 | 4.902° | 18.01359 Å | 100.0% |
| 2 | 9.715° | 9.09702 Å | 6.6% |
| 3 | 10.250° | 8.62280 Å | 17.5% |
| 4 | 13.067° | 6.76950 Å | 11.6% |
| 5 | 13.470° | 6.56813 Å | 19.0% |
| 6 | 13.860° | 6.38399 Å | 10.2% |
| 7 | 16.386° | 5.40510 Å | 17.0% |
| 8 | 18.261° | 4.85414 Å | 9.8% |
| 9 | 19.365° | 4.57983 Å | 29.5% |
| 10 | 20.522° | 4.32423 Å | 16.9% |
| 11 | 21.327° | 4.16282 Å | 28.9% |
| 12 | 21.565° | 4.11735 Å | 29.4% |
| 13 | 24.318° | 3.65705 Å | 21.6% |
| 14 | 27.485° | 3.24246 Å | 6.2% |
| 15 | 29.270° | 3.04864 Å | 9.2% |

The TGA curve obtained from the crystalline form D of Compound 1 adipate is shown in FIG. 14. As shown in FIG. 14, the crystalline form D of Compound 1 adipate exhibited a weight loss of about 14% when heated to 150° C.

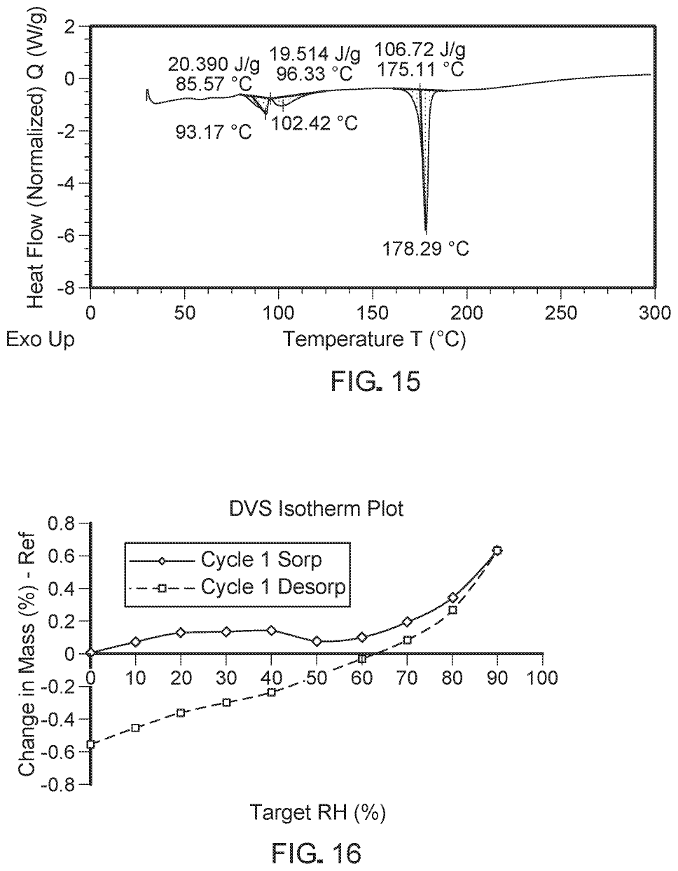

The DSC curve obtained from the crystalline form D of Compound 1 adipate is shown in FIG. 15. As shown in FIG. 15, the crystalline form D of Compound 1 adipate exhibited three endothermic peaks at 86.57° C., 96.33° C., and 175.11° C.

The DVS curve obtained from the crystalline form D of Compound 1 adipate is shown in FIG. 16. As shown in FIG. 16, the crystalline form D of Compound 1 adipate exhibited a weight increase of about 0.34% at 80% RH, which suggests that it has low hygroscopicity.

The above results suggest that the crystalline form D of Compound 1 adipate is a DMAc solvate. In addition, it is believed that the crystalline form D of Compound 1 adipate includes about 1.3 molar DMAc per molar Compound 1 adipate.

### Example 5: Preparation and Characterization of Crystalline Form A of Compound 1 Free Base

Compound 1 HCl (75.5 g) (e.g., obtained by using the method described in Example 5 of U.S. Application Publication No. 2020/0062765) was dissolved in ethanol (604 mL) at 50° C. Sodium hydroxide (68.1 g) was added to the above solution. The mixture was cooled to 1° C. in 1.5 hours and stirred for 18.5 hours. The mixture was then filtered, and the solid thus obtained was washed with a cooled mixture of ethanol (151 mL) and water (151 mL) and dried. The solid thus obtained was confirmed to be the crystalline form A of Compound 1 free base.

The NMR data for the crystalline form A of Compound 1 free base are as follows: 1H NMR (500 MHz, DMSO) δ 1.09-1.10 (d, J=5.0 Hz, 3H), 1.48-1.49 (d, J=5.0 Hz, 3H), 3.16-3.20 (m, 1H), 3.75-3.79 (m, 2H), 4.82-4.86 (m, 1H), 6.76-6.78 (d, J=10.0 Hz, 1H), 6.92-6.94 (m, 2H), 7.01-7.05 (m, 1H), 7.23-7.28 (m, 2H), 7.37-7.42 (m, 1H), 7.62-7.63 (d, J=5.0 Hz, 1H), 7.72-7.75 (m, 4H).

The IR data for the crystalline form A of Compound 1 free base are as follows: IR (cm⁻¹): 3350, 3247, 3055, 2961, 2923, 2864, 1611, 1586, 1349, 829, 819.

A representative XRPD pattern obtained from the crystalline form A of Compound 1 free base is shown in FIG. 17 and the XRPD data are listed in Table 11 below.

TABLE 11

| Index | Angle | d Value | Rel. Intensity |
|---|---|---|---|
| 1 | 8.516° | 10.37390 Å | 100.0% |
| 2 | 11.211° | 7.88611 Å | 1.7% |
| 3 | 12.387° | 7.13994 Å | 8.2% |
| 4 | 12.721° | 6.95283 Å | 69.3% |
| 5 | 14.379° | 6.15479 Å | 5.2% |
| 6 | 15.591° | 5.67900 Å | 5.0% |
| 7 | 16.896° | 5.24328 Å | 28.2% |
| 8 | 17.858° | 4.96295 Å | 32.8% |
| 9 | 19.116° | 4.63887 Å | 36.8% |
| 10 | 19.954° | 4.44602 Å | 34.4% |
| 11 | 21.252° | 4.17730 Å | 14.2% |
| 12 | 21.766° | 4.07970 Å | 9.9% |
| 13 | 22.056° | 4.02683 Å | 12.0% |
| 14 | 24.119° | 3.68690 Å | 0.5% |
| 15 | 25.621° | 3.47406 Å | 16.4% |
| 16 | 26.897° | 3.31196 Å | 3.8% |
| 17 | 27.194° | 3.27657 Å | 4.6% |
| 18 | 27.714° | 3.21626 Å | 8.5% |
| 19 | 28.705° | 3.10737 Å | 7.6% |
| 20 | 29.396° | 3.03588 Å | 4.3% |
| 21 | 30.108° | 2.96575 Å | 2.7% |
| 22 | 31.276° | 2.85758 Å | 2.7% |
| 23 | 32.384° | 2.76232 Å | 1.4% |
| 24 | 33.273° | 2.69051 Å | 1.0% |
| 25 | 33.913° | 2.64115 Å | 11.2% |
| 26 | 34.144° | 2.62383 Å | 15.4% |
| 27 | 35.124° | 2.55282 Å | 5.7% |
| 28 | 36.183° | 2.48047 Å | 1.9% |
| 29 | 37.272° | 2.41047 Å | 2.4% |
| 30 | 40.165° | 2.24327 Å | 2.0% |
| 31 | 40.806° | 2.20949 Å | 1.2% |

The TGA curve obtained from the crystalline form A of Compound 1 free base is shown in FIG. 18. As shown in FIG. 18, the crystalline form A of Compound 1 free base exhibited a weight loss of about 4% when heated to 150° C.

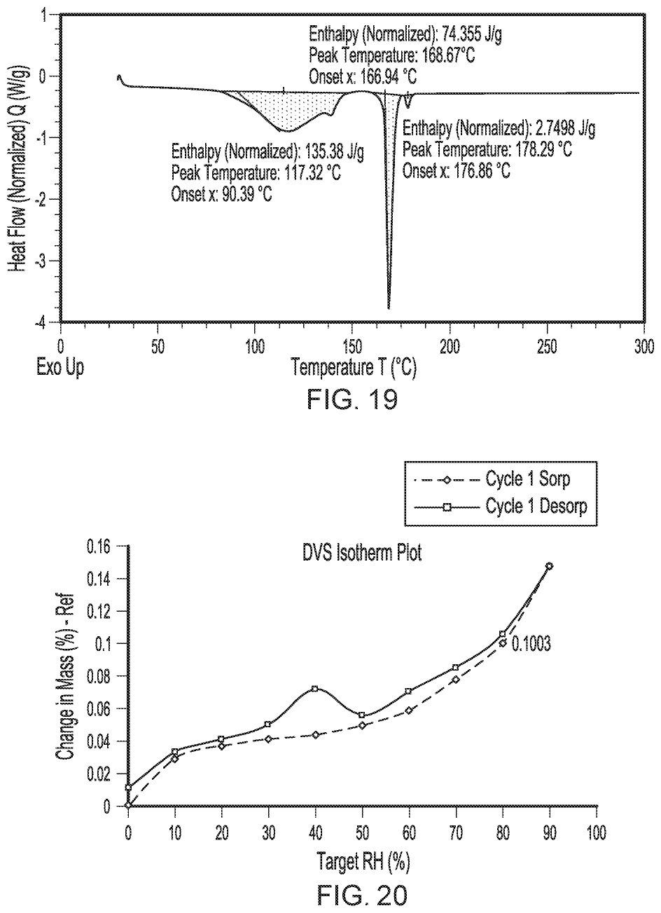

The DSC curve obtained from the crystalline form A of Compound 1 free base is shown in FIG. 19. As shown in FIG. 19, the crystalline form A of Compound 1 free base exhibited three endothermic peaks at 117.32° C., 168.67° C., and 178.29° C.

The DVS curve obtained from the crystalline form A of Compound 1 free base is shown in FIG. 20. As shown in FIG. 20, the crystalline form A of Compound 1 free base exhibited a weight increase of about 0.1% at 80% RH, which suggests that it has almost no hygroscopicity.

The XRPD shows that the crystalline form of the sample before and after the DVS test did not change. On the other hand, after a sample was heated to 155° C., its XRPD shows that the crystalline form changed.

The above results show that the crystalline form A of Compound 1 free base is a hydrate. In addition, it is believed that the crystalline form A of Compound 1 free base includes about 1 molar water per molar Compound 1 free base.

### Example 6: Preparation and Characterization of Crystalline Form B of Compound 1 Free Base

About 50 mg of the crystalline form A of Compound 1 free base was mixed in 1 mL of dichloromethane to form a dispersion. The dispersion thus obtained was stirred at 50° C. for three or seven days to obtain a solid, which was confirmed to be the crystalline form B of Compound 1 free base.

The NMR data for the crystalline form B of Compound 1 free base are as follows: 1H NMR (500 MHz, DMSO) δ 1.25-1.26 (d, J=4.0 Hz, 3H), 1.47-1.49 (d, J=8.0 Hz, 3H), 3.49-3.57 (m, 1H), 3.93-3.98 (m, 1H), 4.06-4.09 (m, 1H), 4.81-4.88 (m, 1H), 6.76-6.78 (d, J=8.0 Hz, 1H), 6.96-6.99 (m, 2H), 7.01-7.06 (m, 1H), 7.22-7.28 (m, 2H), 7.36-7.42 (m, 1H), 7.65-7.67 (d, J=8.0 Hz, 1H), 7.74-7.80 (m, 4H).

The IR data for the crystalline form B of Compound 1 free base are as follows: IR (cm⁻¹): 2960, 2910, 2846, 1624, 1611, 1586, 1335, 829.

A representative XRPD pattern obtained from the crystalline form B of Compound 1 free base is shown in FIG. 21 and the XRPD data are listed in Table 12 below.

TABLE 12

| Index | Angle | d Value | Rel. Intensity |
|---|---|---|---|
| 1 | 6.067° | 14.55558 Å | 100.0% |
| 2 | 9.393° | 9.40725 Å | 60.5% |
| 3 | 9.745° | 9.06833 Å | 33.3% |
| 4 | 11.038° | 8.00937 Å | 24.9% |
| 5 | 11.908° | 7.42558 Å | 20.3% |
| 6 | 12.920° | 6.84647 Å | 18.7% |
| 7 | 13.820° | 6.40229 Å | 33.6% |
| 8 | 14.396° | 6.14750 Å | 14.0% |
| 9 | 15.109° | 5.85911 Å | 20.2% |
| 10 | 17.865° | 4.96093 Å | 8.4% |
| 11 | 18.847° | 4.70460 Å | 34.6% |
| 12 | 19.565° | 4.53356 Å | 16.3% |
| 13 | 20.732° | 4.28092 Å | 34.9% |
| 14 | 21.267° | 4.17438 Å | 66.6% |
| 15 | 26.309° | 3.38472 Å | 9.6% |
| 16 | 29.836° | 2.99214 Å | 10.4% |

The TGA curve obtained from the crystalline form B of Compound 1 free base after removing residual solvent is shown in FIG. 22. As shown in FIG. 22, the crystalline form B of Compound 1 free base exhibited a weight loss of 0.005% (i.e., essentially no weight loss) when heated to 150° C.

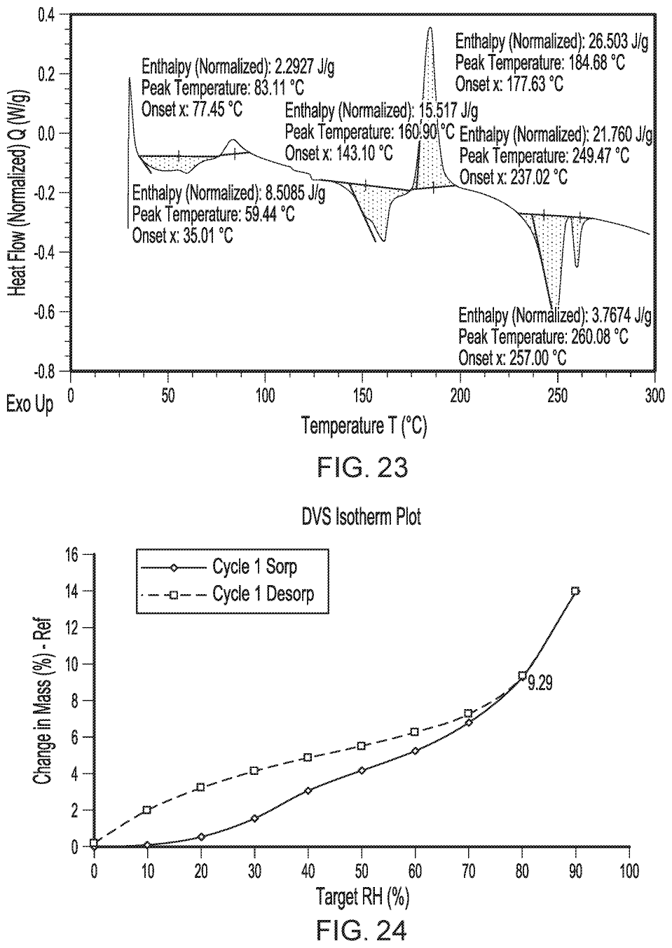

The DSC curve obtained from the crystalline form B of Compound 1 free base is shown in FIG. 23. As shown in FIG. 23, the crystalline form B of Compound 1 free base exhibited an initial melting point of 143.10° C. and a crystal transition peak at 184.68° C.

The DVS curve obtained from the crystalline form B of Compound 1 free base is shown in FIG. 24. As shown in FIG. 24, the crystalline form B of Compound 1 free base exhibited a weight increase of 9.29% at 80% RH, which suggests that it has hygroscopicity.

The XRPD shows that the crystalline form of the sample before and after the removal of residual solvent did not change. On the other hand, the XRPD shows that the crystalline form of the sample before and after the DVS test changed.

The above results suggest that the crystalline form B of Compound 1 free base is an anhydrate.

### Example 7: Preparation and Characterization of Crystalline Form C of Compound 1 Free Base

About 50 mg of the crystalline form A of Compound 1 free base was mixed with 2 mL of dichloromethane. The mixture was heated to 50° C. until the solid was completely dissolved. The solvent was allowed to evaporate at 50° C. to obtain a solid, which was confirmed to be the crystalline form C of Compound 1 free base.

The NMR data for the crystalline form C of Compound 1 free base are as follows: 1H NMR (500 MHz, DMSO) δ 1.08-1.09 (d, J=5.0 Hz, 3H), 1.47-1.48 (d, J=5.0 Hz, 3H), 3.16-3.20 (m, 1H), 3.75-3.80 (m, 2H), 4.81-4.85 (m, 1H), 6.75-6.77 (d, J=10.0 Hz, 1H), 6.91-6.93 (m, 2H), 7.01-7.05 (m, 1H), 7.23-7.28 (m, 2H), 7.37-7.42 (m, 1H), 7.63-7.64 (d, J=5.0 Hz, 1H), 7.72-7.76 (m, 4H).

The IR data for the crystalline form C of Compound 1 free base are as follows: IR (cm⁻¹): 1624, 1610, 1570, 1448, 1457, 1347, 829.

A representative XRPD pattern obtained from the crystalline form C of Compound 1 free base is shown in FIG. 25 and the XRPD data are listed in Table 13 below.

TABLE 13

| Index | Angle | d Value | Rel. Intensity |
|---|---|---|---|
| 1 | 6.661° | 13.25791 Å | 7.3% |
| 2 | 8.790° | 10.05211 Å | 50.7% |
| 3 | 10.562° | 8.36868 Å | 12.2% |
| 4 | 11.455° | 7.71822 Å | 20.2% |
| 5 | 12.053° | 7.33658 Å | 15.7% |
| 6 | 12.428° | 7.11611 Å | 3.8% |
| 7 | 13.193° | 6.70510 Å | 9.7% |
| 8 | 14.490° | 6.10788 Å | 3.4% |
| 9 | 15.259° | 5.80192 Å | 26.0% |
| 10 | 15.584° | 5.68184 Å | 38.7% |
| 11 | 15.876° | 5.57772 Å | 33.7% |
| 12 | 16.200° | 5.46666 Å | 42.4% |
| 13 | 16.501° | 5.38781 Å | 27.6% |
| 14 | 17.438° | 5.08150 Å | 12.4% |
| 15 | 18.598° | 4.76695 Å | 72.5% |
| 16 | 19.239° | 4.60965 Å | 27.5% |
| 17 | 19.788° | 4.48297 Å | 14.8% |
| 18 | 20.198° | 4 39292 Å | 80.5% |
| 19 | 20.627° | 4.30240 Å | 43.4% |
| 20 | 21.075° | 4.21192 Å | 100.0% |
| 21 | 21.658° | 4.09981 Å | 27.9% |
| 22 | 21.913° | 4.05270 Å | 14.4% |
| 23 | 22.540° | 3.94145 Å | 14.5% |
| 24 | 22.921° | 3.87675 Å | 15.4% |
| 25 | 23.341° | 3.80787 Å | 7.2% |
| 26 | 23.698° | 3.75144 Å | 12.5% |
| 27 | 24.088° | 3.69182 Å | 20.5% |
| 28 | 25.200° | 2.53103 Å | 22.4% |
| 29 | 25.635° | 3.47212 Å | 21.7% |
| 20 | 25.933° | 3.43284 Å | 33.1% |
| 31 | 28.276° | 3.15358 Å | 6.8% |
| 32 | 28.986° | 3.07790 Å | 11.1% |
| 33 | 30.085° | 2.96791 Å | 8.4% |
| 34 | 32.888° | 2.72107 Å | 6.5% |
| 35 | 35.258° | 2.54343 Å | 4.8% |

The TGA curve obtained from the crystalline form C of Compound 1 free base is shown in FIG. 26. As shown in FIG. 26, the crystalline form C of Compound 1 free base exhibited a weight loss of 0.107% when heated to 150° C.

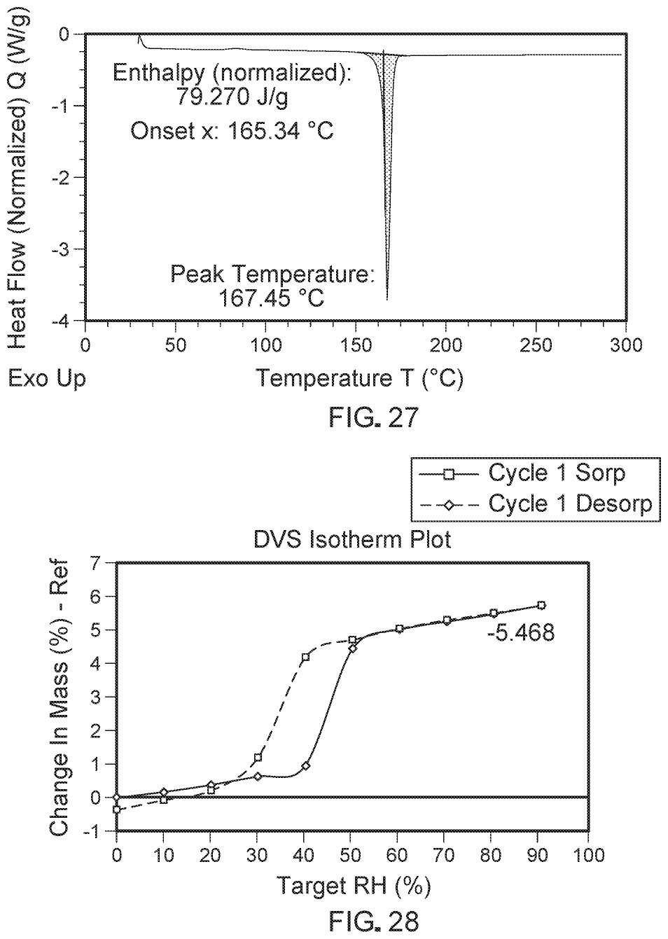

The DSC curve obtained from the crystalline form C of Compound 1 free base is shown in FIG. 27. As shown in FIG. 27, the crystalline form C of Compound 1 free base exhibited an endothermic peak at 167.45° C.

The DVS curve obtained from the crystalline form C of Compound 1 free base is shown in FIG. 28. As shown in FIG. 28, the crystalline form C of Compound 1 free base exhibited a weight increase of 5.468% at 80% RH, which suggests that it has hygroscopicity.

The XRPD of the sample was measured before and after the DVS test. The results show no crystalline form change.

The above results suggest that the crystalline form C of Compound 1 free base is an anhydrate.

### Example 8: Preparation and Characterization of Crystalline Form D of Compound 1 Free Base

100 mg of the crystalline form A of Compound 1 free base was mixed with 1 mL of methanol so that the solid was completely dissolved. Methyl tert-butyl ether (MTBE) (10 mL) was added to the above solution. The solvents in the solution thus obtained were removed by rotary evaporation at 40° C. to obtain a solid, which was confirmed to be the crystalline form D of Compound 1 free base.

The NMR data for the crystalline form D of Compound 1 free base are as follows: 1H NMR (500 MHz, DMSO) δ 1.08-1.10 (d, J=8.0 Hz, 3H), 1.47-1.49 (d, J=8.0 Hz, 3H), 3.13-3.21 (m, 1H), 3.76-3.79 (m, 2H), 4.81-4.88 (m, 1H), 6.75-6.78 (d, J=12.0 Hz, 1H), 6.91-6.94 (m, 2H), 7.00-7.05 (m, 1H), 7.22-7.28 (m, 2H), 7.37-7.42 (m, 1H), 7.60-7.61 (d, J=4.0 Hz, 1H), 7.71-7.75 (m, 4H).

The IR data for the crystalline form D of Compound 1 free base are as follows: IR (cm⁻¹): 1628, 1617, 1570, 1468, 1465, 1348, 1257, 1166, 830.

A representative XRPD pattern obtained from the crystalline form D of Compound 1 free base is shown in FIG. 29 and the XRPD data are listed in Table 14 below.

TABLE 14

| Index | Angle | d Value | Rel. Intensity |
|---|---|---|---|
| 1 | 6.501° | 13.58517 Å | 17.6% |
| 2 | 8.304° | 10.63823 Å | 25.4% |
| 3 | 8.616° | 10.25407 Å | 89.2% |
| 4 | 10.386° | 8.51050 Å | 17.7% |
| 5 | 11.296° | 7.82640 Å | 41.4% |
| 6 | 11.899° | 7.43147 Å | 16.6% |
| 7 | 12.238° | 7.22658 Å | 9.9% |
| 8 | 12.563° | 7.04019 Å | 21.4% |
| 9 | 13.049° | 6.77894 Å | 15.7% |
| 10 | 14.292° | 6.19187 Å | 3.5% |
| 11 | 15.101° | 5.86213 Å | 25.9% |
| 12 | 15.450° | 5.73040 Å | 63.2% |
| 13 | 15.697° | 5.64082 Å | 43.0% |
| 14 | 16.020° | 5.52773 Å | 51.8% |
| 15 | 16.258° | 5.44758 Å | 28.2% |
| 16 | 16.605° | 5.33444 Å | 3.3% |
| 17 | 17.293° | 5.12364 Å | 6.0% |
| 18 | 17.472° | 5.07153 Å | 8.7% |
| 19 | 18.028° | 4.91645 Å | 54.1% |
| 20 | 18.437° | 4.80835 Å | 85.4% |
| 21 | 19.090° | 4.64523 Å | 30.3% |
| 22 | 19.671° | 4.50931 Å | 28.3% |
| 23 | 20.060° | 4.42272 Å | 72.1% |
| 24 | 20.473° | 4.33448 Å | 36.7% |
| 25 | 20.893° | 4.24824 Å | 100.0% |
| 26 | 21.486° | 4.13234 Å | 23.0% |
| 27 | 21.985° | 4.03970 Å | 39.3% |
| 28 | 22.353° | 3.97405 Å | 5.8% |
| 29 | 22.774° | 3.90139 Å | 5.8% |
| 30 | 23.637° | 3.76091 Å | 10.6% |
| 31 | 23.976° | 3.70848 Å | 29.5% |
| 32 | 25.171° | 3.53511 Å | 34.1% |
| 33 | 25.489° | 3.49175 Å | 21.8% |
| 34 | 25.758° | 3.45583 Å | 28.7% |
| 35 | 28.829° | 3.09435 Å | 8.4% |
| 36 | 29.249° | 3.05077 Å | 4.0% |
| 37 | 29.929° | 2.98302 Å | 4.8% |
| 38 | 35.102° | 2.55434 Å | 2.7% |

The TGA curve obtained from the crystalline form D of Compound 1 free base is shown in FIG. 30. As shown in FIG. 30, the crystalline form D of Compound 1 free base exhibited a weight loss of 1.263% when heated to 150° C.

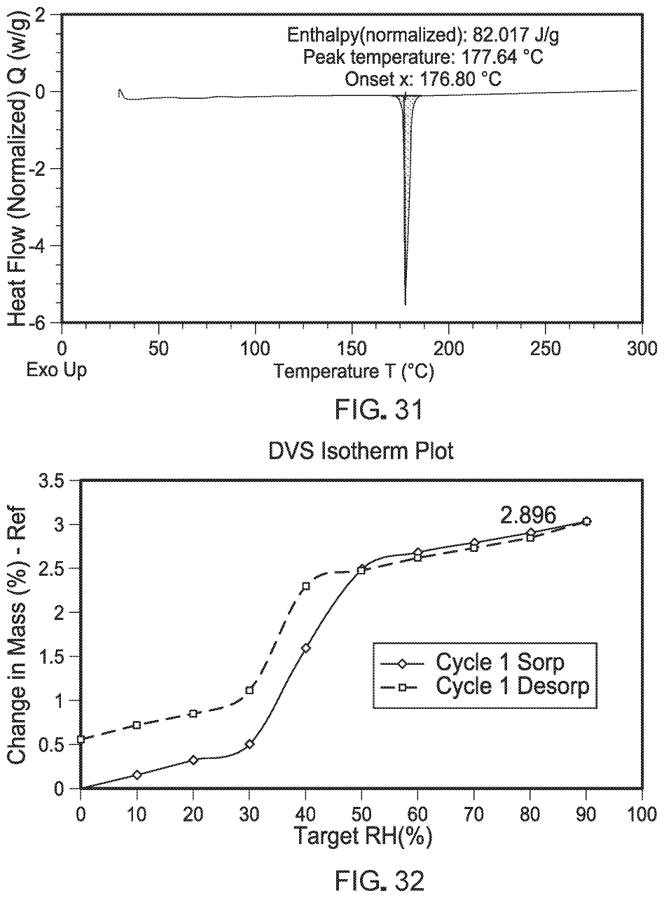

The DSC curve obtained from the crystalline form D of Compound 1 free base is shown in FIG. 31. As shown in FIG. 31, the crystalline form D of Compound 1 free base exhibited an endothermic peak at 177.64° C.

The DVS curve obtained from the crystalline form D of Compound 1 free base is shown in FIG. 32. As shown in FIG. 32, the crystalline form D of Compound 1 free base exhibited a weight increase of 2.896% at 80% RH, which suggests that it has some hygroscopicity.

The XRPD shows that the crystalline form of the sample before and after the removal of residual solvent changed. On the other hand, the XRPD shows that the crystalline form of the sample before and after the DVS test did not change.

The above results suggest that the crystalline form D of Compound 1 free base is a solvate/hydrate mixture.

Other embodiments are within the scope of the following claims.

1. A crystalline form of 3-{4-[(2R)-2-aminopropoxy]phenyl}-N-[(1R)-1-(3-fluorophenyl)ethyl]imidazo[1,2-b]pyridazin-6-amine (Compound 1) adipate, wherein the crystalline form is:
form A having a tetragonal crystal system, the space group is P41212, and the unit cell parameters are a=b=9.63 (1) Å, c=61.14 (2) Å, α=β=γ=90°, and V=5666 (5) Å³;
form B exhibiting an X-ray powder diffraction (XRPD) pattern that comprises at least one diffraction peak having a diffraction angle 2θ selected from the group consisting of 5.2±0.2°, 7.2±0.2°, and 20.9±0.2° obtained by using CuKα radiation;
form C exhibiting an XRPD pattern that comprises at least one diffraction peak having a diffraction angle 2θ selected from the group consisting of 5.7±0.2°, 21.0±0.2°, and 23.2±0.2° obtained by using CuKα radiation; or
form D exhibiting an XRPD pattern that comprises at least one diffraction peak having a diffraction angle 2θ selected from the group consisting of 8.6±0.2°, 18.4±0.2°, and 20.9±0.2° obtained by using CuKα radiation.

2-4. (canceled)

5. The crystalline form of claim 1, wherein the crystalline form is form B.

6-9. (canceled)

10. A method of preparing the crystalline form B of claim 5, the method comprising:
dissolving an amorphous form of Compound 1 adipate in a solvent comprising dichloromethane and methanol to form a solution; and
evaporating the solvent to obtain the form B of Compound 1 adipate.

11. The crystalline form of claim 1, wherein the crystalline form is form C.

12-15. (canceled)

16. A method of preparing the crystalline form C of claim 11, the method comprising:
dissolving an amorphous form of Compound 1 adipate in ethanol to form a solution;
adding acetone into the solution; and
removing ethanol and acetone by evaporation to obtain the form C of Compound 1 adipate.

17. The crystalline form of claim 1, wherein the crystalline form is form D.

18-20. (canceled)

21. A pharmaceutical composition, comprising:
the form D of claim 17; and
a pharmaceutically acceptable carrier.

22. A method of preparing the crystalline form D of claim 17, the method comprising:
dissolving an amorphous form of Compound 1 adipate in dimethylacetamide to form a solution;
adding acetone into the solution; and
removing dimethylacetamide and acetone by evaporation to obtain the form D of Compound 1 adipate.

23. A crystalline form of 3-{4-[(2R)-2-aminopropoxy]phenyl}-N-[(1R)-1-(3-fluorophenyl)ethyl]imidazo[1,2-b]pyridazin-6-amine (Compound 1) free base, wherein the crystalline form is:
form A exhibiting an X-ray powder diffraction (XRPD) pattern that comprises at least one diffraction peak having a diffraction angle 2θ selected from the group consisting of 8.5±0.2°, 12.7±0.2°, and 19.1±0.2° obtained by using CuKα radiation;
form B exhibiting an XRPD pattern that comprises at least one diffraction peak having a diffraction angle 2θ selected from the group consisting of 6.1±0.2°, 9.4±0.2°, and 21.3±0.2° obtained by using CuKα radiation;
form C exhibiting an XRPD pattern that comprises at least one diffraction peak having a diffraction angle 2θ selected from the group consisting of 18.6±0.2°, 20.2±0.2°, and 21.1±0.2° obtained by using CuKα radiation; or
form D exhibiting an XRPD pattern that comprises at least one diffraction peak having a diffraction angle 2θ selected from the group consisting of 8.6±0.2°, 18.4±0.2°, and 20.9±0.2° obtained by using CuKα radiation.

24-27. (canceled)

28. A method of preparing the crystalline form A of claim 23, the method comprising:
mixing a base with a solution comprising Compound 1 hydrochloride in water and an alcohol to obtain the form A of Compound 1 free base.

29. (canceled)

30. The crystalline form of claim 23, wherein the crystalline form is form B.

31-34. (canceled)

35. A method of preparing the crystalline form B of claim 30, the method comprising:
dispersing a form A of Compound 1 free base in dichloromethane to form a dispersion; and
stirring the dispersion at a temperature from about 45° C. to about 55° C. to obtain the crystalline form B of Compound 1 free base.

36. The crystalline form of claim 23, wherein the crystalline form is form C.

37-40. (canceled)

41. A method of preparing the crystalline form C of claim 36, the method comprising:
dissolving a form A of Compound 1 free base in dichloromethane to form a solution; and
removing dichloromethane by evaporation to obtain the form C of Compound 1 free base.

42. The crystalline form of claim 23, wherein the crystalline form is form D.

43-46. (canceled)

47. A method of preparing the crystalline form D of claim 42, the method comprising:
dissolving a form A of Compound 1 free base in methanol to form a solution;
adding methyl tert-butyl ether to the solution; and
removing methanol and methyl tert-butyl ether by evaporation to obtain the form D of Compound 1 free base.

48. A method of treating cancer, comprising administering to a subject in need thereof a therapeutically effective amount of the composition of claim 1.

49-52. (canceled)

53. The crystalline form of claim 1, wherein the crystalline form is form A.

54. A pharmaceutical composition, comprising:
a crystalline form of claim 1; and
a pharmaceutically acceptable carrier.

55. A pharmaceutical composition, comprising:
a crystalline form of claim 23; and
a pharmaceutically acceptable carrier.

56. A method of treating cancer, comprising administering to a subject in need thereof a therapeutically effective amount of the composition of claim 23.

57. A method of preparing a crystalline form of claim 1, the method comprising:
mixing an amorphous form of Compound 1 adipate with a solvent; and
adding an anti-solvent into the mixture to obtain the form A of Compound 1 adipate.
